(19) United States
(12) Patent Application Publication
(10) Pub. No.: US 2024/0252444 A1
(43) Pub. Date: Aug. 1, 2024

(54) ENCAPSULATION OF LIVE MICROORGANISMS

(71) Applicant: Tagra Biotechnologies Ltd., Natania (IL)

(72) Inventors: Danny GOLDSTEIN, Kibbutz Dafna (IL); Shaher DUCHI, Kfar Rama (IL); Akram HAJOUJ, Kaukab Abu al-Hija (IL); Revital SCHIMMERLING, Ramat Yishay (IL); Rawya SHKOOR, Kfar Metzar (IL)

(73) Assignee: Tagra Biotechnologies Ltd., Natania (IL)

(21) Appl. No.: 18/564,127

(22) PCT Filed: May 27, 2022
(86) PCT No.: PCT/IL2022/050568

§ 371 Date: Nov. 27, 2023

Related U.S. Application Data
(60) Provisional application No. 63/193,703, filed on May 27, 2021.

(51) Int. Cl. A61K 9/50; A23L 33/135; A61K 8/11; A61K 8/99; A61K 35/741

(52) U.S. Cl. CPC A61K 9/5042; A23L 33/135; A61K 8/11; A61K 8/99; A61K 35/741

(57) ABSTRACT

A microcapsule comprising an inner core that comprises a living microorganism enveloped by an outer shell formed of a wall-forming polymeric material are provided. Processes of preparing the microcapsule and products containing same are also provided.

### RELATED APPLICATION/S

This application claims the benefit of priority under 35 USC § 119(e) of U.S. Provisional Patent Application No. 63/193,703 filed May 27, 2021, the contents of which are incorporated herein by reference in their entirety.

### FIELD AND BACKGROUND OF THE INVENTION

The present invention, in some embodiments thereof, relates to encapsulation and, more particularly, but not exclusively, to microcapsules encapsulating a living microorganism, to processes of preparing same and to products and/or formulations such as foods, pharmaceutical, cosmetics and agricultural products and/or formulations containing same.

Probiotics are ‘live microorganisms’ or ‘live microbial feed supplements’ that offer health benefits to their host and include, inter alia, bacteria from the Lactobacillus and Bifidobacterium genera. Exemplary Lactobacillus species used as probiotics include L. delbreuckii subspecies bulgaricus, L. acidophilus, L. casei, L. germentum, L. plantarum, L. brevis, L. cellobious, L. lactis and L. reuteri.

In foods and oral formulations, the probiotics are generally used as a means for restoring microbial balance, particularly in the gastrointestinal tract. This approach appears particularly significant since the intestinal microbiota is involved in physiological balance and in the intestinal development and maturation of the host immune.

In the last years an increasing interest has been focused on the possible use of ingested probiotics for treating inflammatory and allergic conditions by using specific strains that are able to modulate the immune system at the local and systemic levels. Recently, the impact of probiotics on dermatology has been suggested. Indeed, an emerging approach to help preventing and treating skin conditions, including the external signs of aging, acne, rosacea, yeast and bacterial infections, psoriasis, and dermatitis, is represented by topical probiotics, as shown by the growing marketplace for topical probiotic formulations available for skin care and antiaging benefits. The potential benefits of skin probiotics is believed to depend on how each species or strain is selected as the specific mechanisms underlying a specific effect on the healthy or disturbed skin.

Clinical and experimental researches extensively document that beyond probiotic capacity to beneficially influence the intestinal functions, they can exert their benefits at the skin level.

Scientific, and evidence-based reports have strengthen the assumption that certain probiotics can contribute to modulate cutaneous microflora, lipid barrier, and skin immune system, leading to the preservation of the skin homeostasis. New strategies that are based on a topical approach for the prevention and treatment of cutaneous disorders using live microorganisms are therefore being sought for. The use of probiotic bacteria and lysates thereof in medical and cosmetic treatment is described, for example, in WO 2012/150269 and WO 2013/153358.

In addition, agricultural biological agents have been considered recently as an attractive alternative to agrochemical seed treatments. Microbial agents are one class of such agricultural biologicals that include naturally occurring microorganisms that have been found to promote plant growth and/or control of pests and weeds. As more impacts from agrochemical usage are identified, agricultural biologicals are becoming more attractive due to their natural latency in the environment. Despite these advantages, the use of agricultural biologicals is limited due to poor stability and efficacy. As living organisms, they are less robust than agrochemicals. Their efficacy can be altered by many factors, including long storage times, changes in temperature and humidity, and rapid desiccation from typical seed treatment processes, and for formulations that can stabilize microbial agricultural biologicals against such factors are sought for.

Probiotics are sensitive to various environmental conditions and typically lack the ability to survive for long periods of time in “high acid’ formulation preparations (foods and beverage products like citrus fruit juices, tomato sauce, etc. and cosmetic products). For example, in water-based cosmetic products probiotics are sensitive to numerous conditions, including, e.g., low pH, high acid content, high water activity, heat, air, light, and the inherent presence of polyphenols, as well as other environmental influences. Thus, the viability (measured in colony forming units or CFU), and therefore the efficacy, in products supplemented with probiotics is often substantially reduced.

Encapsulation or microencapsulation is a process of entrapping substances (e.g., active agents) within a carrier (typically a solid carrier) material.

A microencapsulation method refers to a physicochemical or mechanical process for entrapping a substance in a material to produce particles with diameters of a few microns to a few millimeters. Microcapsules are small particles that contain an active agent or core material surrounded by a coating or shell. Encapsulation shell materials include a variety of polymers, carbohydrates, fats and waxes, depending on the core material to be protected.

Microencapsulation is a useful tool for incorporating living microorganisms into, for example, foods, pharma, cosmetics and agricultural formulations, to protect, to extend their storage life, and to convert them into a powder form for convenient use. Encapsulation of living microorganisms can also promote controlled release and optimize delivery to the site of action, thereby potentiating the efficacy of the respective strain. This process can also prevent microorganisms from multiplying in the formulation that would otherwise change their aesthetic and sensory characteristics.

U.S. Pat. Nos. 6,932,984, 7,838,037, WO 2009/138978, WO 2015/132791, WO 2015/132792 and WO 2017/037716, all by the present assignee, disclose microcapsules designed to rupture by a slight mechanical action such as rubbing or pressing on the skin, and thereby immediately release their encapsulated content. These microcapsules are prepared by the solvent removal method using non-chlorinated organic solvents.

The majority of encapsulated microorganism focus on gelled capsules comprising proteins and/or polysaccharides such as alginate and chitosan (EP Patent Application Publication No. 0152898; WO 2017/087939; U.S. Patent No. 2018/0360707; and U.S. Patent Application Publication No. 2012/0263826).

The selection of the best encapsulation technology for living microorganisms such as probiotics should take into consideration numerous aspects to guarantee the survival of the microorganisms during the encapsulation process, in storage conditions and during application, as well as the release mechanism in the specific desired area of application. The most problematic issues encountered in probiotic encapsulation typically include keeping the microorganisms alive during manufacturing and storage and releasing the microorganisms onsite while maintaining their efficacy.

Viable bacteria were encapsulated in a gelled substance which was designed to release the bacteria upon prolonged exposure to moisture in mucous membranes (WO 1996/038159). Further coating by materials such as acrylic/methacrylic acid ester copolymer and Eudragit™ was suggested to enhance the survival of probiotics.

WO 2015/019307 discloses a probiotic microcapsule comprising a biopolymer and plant protein shell, and designed to release its contents into the GI tract once triggered by an external stimuli such as temperature, pH or exposure to certain enzymes.

U.S. Patent Application Publication No. 2020/0108105 discloses probiotic microcapsules made of a lipid, wax and/or silicone matrix, which may be further shelled by various materials. The capsule was designed to have a melting or softening temperature in a range of 20-43° C., thus allowing microorganisms to release upon contact with skin and/or mucous membranes of a subject.

U.S. Pat. No. 10,548,844 discloses a formulation of encapsulated microorganisms comprising mucoadhesive polymers (e.g., cellulose and cellulose derivatives), which are designed to release the microorganisms upon exposure to low pH within the GI tract. The microcapsule was prepared from 3 bilayers of chitosan and alginate.

Additional Background Art includes: Dixit Y, Wagle A, Vakil B (2016) Patents in the Field of Probiotics, Prebiotics, Synbiotics: A Review. J Food Microbiol Saf Hyg 1: 111; U.S. Pat. Nos. 8,142,831 and 10,479,963; U.S. Patent Application Publication No. 2005/0266069; EP Patent No. 3210612; WO 2006/136196; and WO 2011/022790.

### SUMMARY OF THE INVENTION

There is an unmet need for probiotic compositions/products capable of maintaining high viability and stability of the microorganisms under industrial and physiological conditions.

The present inventors have designed and successfully practiced a novel methodology for encapsulating living microorganisms, such as probiotic microorganisms, in the presence of an organic solvent, while maintaining the viability and activity of the encapsulated microorganism.

According to an aspect of some embodiments of the present invention there is provided a microcapsule comprising an inner core enveloped by an outer shell formed of a wall-forming polymeric material, wherein the inner core comprises a living microorganism.

According to some of any of the embodiments described herein, the microcapsule is characterized by: (a) the wall-forming polymeric material having a positive log P value; and/or (b) the microcapsule is rupturable by a mechanical action of rubbing or pressing with a human hand; and/or (c) the wall-forming polymeric material being dissolvable at a concentration of at least 100 grams per liter in a partially water-miscible organic solvent that is compatible with the microorganism.

According to some of any of the embodiments described herein, the microcapsule is characterized by the (a), (b) and (c).

According to some of any of the embodiments described herein, the outer shell further comprises a fatty substance.

According to some of any of the embodiments described herein, the fatty substance is selected from the group consisting of a wax, a lipid and/or an oil.

According to some of any of the embodiments described herein, the fatty substance is a naturally occurring and/or biocompatible fatty substance.

According to some of any of the embodiments described herein, the fatty substance is a natural wax.

According to some of any of the embodiments described herein, the fatty substance comprises soy wax.

According to some of any of the embodiments described herein, an amount of the fatty substance ranges from about 1 to about 20, or from about 1 to about 10, or from about 5 to about 15, % by weight of the total weight of the microcapsule.

According to some of any of the embodiments described herein, an amount of the wall-forming polymeric material ranges from about 10 to about 95, or from about 20 to about 80, or from about 20 to about 50, % by weight of the total weight of the microcapsule.

According to some of any of the embodiments described herein, an amount of the living microorganism ranges from about 1 to about 30, or from about 5 to about 25, or from about 10 to about 20, % by weight of the total weight of the microcapsule.

According to some of any of the embodiments described herein, the outer shell further comprises a plasticizer.

According to some of any of the embodiments described herein, the plasticizer is a naturally-occurring and/or biocompatible plasticizer.

According to some of any of the embodiments described herein, the plasticizer is selected from a group consisting of triethyl citrate, tricaprylin (caprylic triglyceride), trilaurin, tripalmitin, triacetin, acetyltriethyl citrate, paraffin oil, and any combination thereof.

According to some of any of the embodiments described herein, the plasticizer is caprylic triglyceride.

According to some of any of the embodiments described herein, the wall-forming polymer comprises a naturally occurring polymer and/or biocompatible polymer.

According to some of any of the embodiments described herein, the wall-forming polymer is selected from a group consisting of polyacrylates, polymethacrylates, low molecular weight poly(methyl methacrylate)-co-(methacrylic acid) (e.g., 1:0.16), poly(ethyl acrylate)-co-(methyl methacrylate)-co-(trimethylammonium-ethyl methacrylate chloride) (e.g., 1:2:0.1) (also known as Eudragit® RSPO), poly(butyl methacrylate)-co-(2-dimethylaminoethyl methacrylate)-co-(methyl methacrylate) (e.g., 1:2:1), poly(styrene)-co-(maleic anhydride), copolymer of octylacrylamide, cellulose ethers, cellulose esters, poly(ethylene glycol)-block-poly(propylene glycol)-block-poly(ethylene glycol), PLA (poly(lactic acid), PGA (poly(glycolide), PLGA (poly(lactide)-co-poly(glycolide) or any combination thereof.

According to some of any of the embodiments described herein, the wall-forming polymer comprises a cellulose ester.

According to some of any of the embodiments described herein, the cellulose ester comprises from 1 to 10% free hydroxy groups.

According to some of any of the embodiments described herein, the wall-forming polymer is or comprises cellulose acetate butyrate.

According to some of any of the embodiments described herein, the cellulose acetate butyrate comprises from 10 to 50% butyryl groups.

According to some of any of the embodiments described herein, the wall-forming polymeric material has a log P value in a range of from 2 to 20, or from 2 to 10.

According to some of any of the embodiments described herein, the wall-forming polymeric material is dissolvable in ethyl acetate at a concentration of from 100 grams to 1,000 grams per liter, or from 100 grams to 800 grams per liter.

According to some of any of the embodiments described herein, the living microorganism comprises a probiotic microorganism.

According to some of any of the embodiments described herein, the probiotic microorganism is beneficial for use in a pharmaceutical product and is selected from the group consisting of Bacillus coagulans GBI-30, 6086, Bacillus subtilis var natt, Bacillus sp., Bifidobacterium Lafti®™ B94, Bifidobacterium bifidum, Bifidobacterium bifidum rosell-71, Bifidobacterium breve, Bifidobacterium breve Rosell-70, Bifidobacterium infantis, Bifidobacterium lactis, Bifidobacterium longum, Bifidobacterium longum Rose-175, Bifidobacterium animalis, Bifidobacterium animalis subsp. lactis BB-12, Bifidobacterium animalis subsp. lactis HN019, Bifidobacterium infantis 35624, Escherichia coli M-17, Escherichia coli Nissle 1917, Lactobacillus acidophilus, Lactobacillus acidophilus Lafti®™ L10, Lactobacillus casei Lafti®™ L26, Lactobacillus brevis, Lactobacillus bulgaricus, Lactobacillus casei, Lactobacillus gasseri, Lactobacillus paracasei, Lactobacillus plantarum, Lactobacillus pentosus, Lactobacillus reuteri ATTC 55730 (Lactobacillus reuteri SD2112), Lactobacillus rhamnosus, Lactobacillus salivarius, Lactobacillus delbrueckii, Lactobacillus fermentum, Lactococcus lactis; Lactococcus lactis subsp, Lactococcus lactis Rosell-1058, Lactobacillus paracasei St11, Lactobacillus paracasei NCC2461, Lactobacillus fortis, Lactobacillus johnsonii La1, Lactobacillus rhamnosus Rosell-11, Lactobacillus acidophilus Rosell-52, Streptococcus thermophilus, Streptococcus sp., Diacetylactis spp., Saccharomyces cerevisiae, Enterococcus spp., Pediococcus spp., Propionibacterium spp., Peptosteptococcus spp., and any combination or genetically-modified variants thereof.

According to some of any of the embodiments described herein, the probiotic microorganism is beneficial for use in a cosmetic product and is selected from a group consisting of Bifidobacterium spp., Lactobacillus sp., Lactobacillus curvatus, Lactobacillus paracasei, Lactobacillus pentosus, Lactobacillus plantarum, Bacillus spp., Streptococcus spp., Micrococcus spp., Saccharomyces sp., Saccharomyces boulardii, Staphylococcus epidermidis, Staphylococcus salivarius, Lactococcus sp. HY449, Streptococcus thermophiles, Enterococcus faecalis, and any combination or genetically-modified variants thereof.

According to some of any of the embodiments described herein, the probiotic microorganism is beneficial for use in a cosmeceutical product and is selected from a group consisting of Staphylococcus epidermidis, Staphylococcus salivarius, Lactococcus sp. HY449, Streptococcus thermophiles, Enterococcus faecalis, Lactobacillus plantarum, Lactobacillus pentosus, and any combination or genetically-modified variants thereof.

According to some of any of the embodiments described herein, the probiotic microorganism is beneficial for use in a food product and is selected from a group consisting of Bacillus coagulans GBI-30, 6086, Bacillus subtilis var. natto, Bifidobacterium Lafti®™ B94, Bifidobacterium bifidum, Bifidobacterium bifidum rosell-71, Bifidobacterium breve, Bifidobacterium breve Rosell-70, Bifidobacterium infantis, Bifidobacterium lactis, Bifidobacterium longum, Bifidobacterium longum Rose-175, Bifidobacterium longum BB 536, Bifidobacterium animalis, Bifidobacterium animalis subsp. animalis, Bifidobacterium animalis subsp. lactis BB-12, Bifidobacterium animalis subsp. lactis HN019, Bifidobacterium infantis 35624, Escherichia coli M-17, Escherichia coli Nissle 1917, Lactobacillus acidophilus, Lactobacillus acidophilus LA5, Lactobacillus acidophilus Rosell-52, Lactobacillus acidophilus Lafti®™ L10, Lactobacillus casei, Lactobacillus casei Lafti®™ L26, Lactobacillus casei L19, Lactobacillus casei F19, Lactobacillus casei 431, Lactobacillus brevis, Lactobacillus bulgaricus, Lactobacillus gasseri, Lactobacillus paracasei, Lactobacillus plantarum, Lactobacillus plantarum 299v, Lactobacillus reuteri, Lactobacillus reuteri ATTC 55730, Lactobacillus rhamnosus, Lactobacillus rhamnosus Rosell-11, Lactobacillus rhamnosus LGG, Lactobacillus rhamnosus LB21, Lactobacillus rhamnosus 271, Lactobacillus salivarius, Lactobacillus delbrueckii, Lactobacillus fermentum, Lactococcus lactis, Lactococcus lactis subsp. lactis L1A, Lactococcus lactis Rosell-1058, Lactobacillus paracasei St11, Lactobacillus paracasei NCC2461, Lactobacillus fortis, Lactobacillus johnsonii, Lactobacillus johnsonii La1, Lactobacillus pentosus, Streptococcus thermophilus, Diacetylactis, Saccharomyces cerevisiae, and any combination or genetically-modified variants thereof.

According to some of any of the embodiments described herein, the probiotic microorganism is beneficial for use in an agricultural product and is selected from a group consisting of Alcaligenes faecalis, Bacillus sp., Bacillus subtilis, Bacillus megaterium, Bacillus velezensis, Bacillus okhensis, Bacillus polymyxa, Bacillus subtilis, Exophiala sp., Fusarium culmorum, Halomonas sp., Lactobacillus plantarum, Novosphingobium sp., Penicillium minioluteum, Penicillium sp., Phoma glomerata, Pseudomonas sp., Pseudomonas fluorescens, Pseudomonas alcaligenes, Pseudomonas chlororaphis, Pseudomonas mendocina, Pseudomonas spp., Pantoea sp., Sinorhizobium meliloti, Trichoderma longibrachiatum, Trichoderma harzianum, Trichoderma harzianum subsp. T22, Enterobacter sp., Arthrobacter sp., Enterobacter ludwigii, Lactobacillus plantarum, Ampelomyces quisqualis M-10, Azospirillum lipoferum, Azospirillum spp., Bacillus subtilis GB03, Bradyrhizobium japonicum, Bacillus pumilus GB34, Coniothyrium minitans, Delftia acidovorans, Phlebiopsis gigantean, Streptomyces griseoviridis K61, Ralstonia sp., Escherichia fergusonii, Acinetobacter calcoaceticus, Salmonella enterica, Brevibacillus choshinensis, Pectobacterium carotovorum, Microbacterium testaceum, Mycobacterium phlei, Cedecea davisae, Curtobacterium M84, Arthrobacter oxidans BB1, Rhizobium spp., Acetobacter spp., Azotobacter spp., Colletotrichum spp., Agrobacterium tumefaciens, Zhinguelliuella spp., Brachybacterium saurashtrense, Vibrio spp., Brevibacterium casei, Haererohalobacter spp., Azotobacter chroococcum W5, Mesorhizobium ciceri F75, Pseudomonas striata P27, Serratia marcescens L11, Anabaena torulosa, Pseudomonas GGRJ21, Bacillus cereus Pb25, Bacillus amyloliquefaciens RWL-1, and any combination or genetically-modified variants thereof.

According to some of any of the embodiments described herein, a viability and/or activity of the living microorganism is at least 50, or at least 60, or at least 70, or at least 80, or at least 90, %, relative to a free, non-encapsulated form of the living microorganism when tested under the same experimental conditions.

According to some of any of the embodiments described herein, the microcapsule is a single-layer microcapsule.

According to some of any of the embodiments described herein, the outer shell further comprises a fatty substance and a plasticizer, the wall-forming polymeric material comprises a naturally occurring polymer, an amount of the fatty substance ranges from about 1 to about 20% by weight of the total weight of the microcapsule; an amount of the wall-forming polymeric material ranges from about 10 to about 95% by weight of the total weight of the microcapsule; and an amount of the living microorganism ranges from about 1 to about 30% by weight of the total weight of the microcapsule.

According to some of any of the embodiments described herein, the outer shell further comprises a wax and a plasticizer; the wall-forming polymeric material comprises a cellulose ester; an amount of the fatty substance ranges from about 5 to about 15% by weight of the total weight of the microcapsule; an amount of the wall-forming polymeric material ranges from about 15 to about 80% by weight of the total weight of the microcapsule; and an amount of the living microorganism ranges from about 5 to about 25% by weight of the total weight of the microcapsule.

According to some of any of the embodiments described herein, the outer shell further comprises soy wax and caprylic triglyceride; the wall-forming polymeric material comprises cellulose acetate butyrate; an amount of the fatty substance ranges from about 5 to about 10% by weight of the total weight of the microcapsule; an amount of the wall-forming polymeric material ranges from about 20 to about 50% by weight of the total weight of the microcapsule; and an amount of the living microorganism ranges from about 10 to about 20% by weight of the total weight of the microcapsule.

According to an aspect of some embodiments of the present invention there is provided a composition comprising a plurality of microcapsules, at least a portion, or each, of the microcapsules are microcapsules as described herein in any of the respective embodiments and any combination thereof. According to some of any of the embodiments described herein, the composition is in a form of a powder.

According to an aspect of some embodiments of the present invention there is provided a product comprising the composition as described herein in any of the respective embodiments.

According to some of any of the embodiments described herein, the product a pharmaceutical product which comprises microorganisms having a therapeutic effect, a cosmetic product which comprises microorganisms having a cosmetic effect, a cosmeceutical product which comprises microorganisms having a cosmeceutical effect, a food product which comprises microorganisms having a beneficial physiological effect, or an agricultural product which comprises microorganisms having an agricultural effect.

Further according to embodiments of the present invention there are provided uses of a product as described herein in a respective application. For example, a pharmaceutical product as described herein is for use in the treatment of a medical condition that is treatable by a microorganism having a respective therapeutic effect. A cosmetic or cosmeceutical product as described herein is for use in the treatment of a skin or mucosal condition that is treatable by a respective microorganism. An agricultural product as described herein is for use in controlling growth of a crop.

According to an aspect of some embodiments of the present invention there is provided a process of preparing a microcapsule that comprises a living microorganism. According to some of any of the embodiments described herein, the process is based on the solvent-removal method of preparing microcapsules. According to some of any of the embodiments described herein, the process is for preparing a microcapsule as described herein in any of the respective embodiments.

According to some of any of the embodiments described herein, the process comprises: (a)
contacting an organic phase comprising the partially water-miscible organic solvent, the wall-forming polymer, the living microorganism, and an emulsifier, with an aqueous continuous phase saturated with the partially water-miscible organic solvent, to thereby obtain an emulsion; and (b) adding to the emulsion an amount of water which initiates extraction of the partially water-miscible organic solvent from the emulsion, thereby obtaining the microcapsules encapsulating the living microorganisms.

According to some of any of the embodiments described herein the partially water-miscible organic solvent is compatible with the microorganism.

According to some of any of the embodiments described herein the partially water-miscible organic solvent is or comprises ethyl acetate.

According to some of any of the embodiments described herein the organic phase further comprises a fatty substance.

According to some of any of the embodiments described herein, the fatty substance is selected from the group consisting of a wax, a lipid and/or an oil.

According to some of any of the embodiments described herein the fatty substance is a biocompatible fatty substance.

According to some of any of the embodiments described herein the fatty substance is or comprises a natural wax.

According to some of any of the embodiments described herein the fatty substance is or comprises soy wax.

According to some of any of the embodiments described herein the organic phase further comprises a plasticizer.

According to some of any of the embodiments described herein, the process further comprises isolating the microcapsules.

According to some of any of the embodiments described herein, the process further comprises washing and sifting the microcapsules.

According to some of any of the embodiments described herein, the process further comprises dehydrating the microcapsules to obtain dehydrated microcapsules.

According to some of any of the embodiments described herein the dehydration is effected by freeze drying, heat drying, vacuum drying or any combination thereof.

According to some of any of the embodiments described herein, the dehydrated microcapsules are in the form of a powder.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B present light microscopy images of dried microcapsules (FIG. 1A) and microcapsules during extraction stage (FIG. 1B).

FIG. 2 presents comparative plots showing the change in pH over time of an aqueous solution containing a free probiotic microorganism and a probiotic microorganism after encapsulation and breakage of the microcapsules containing same.

FIG. 3 is a table presenting viability of L. plantarum and L. acidofulus following 30 seconds of vortexing with ethyl acetate and a 10-minute incubation.

FIG. 4 is a picture of a plate with a sample from L. plantarum following a 2-hour incubation with MRS agar and Tween® 80. The sample contained 93188 CFU per gram microcapsule following the 2-hour incubation.

FIGS. 5A and 5B are photos taken from scanning electron microscopy (SEM) analysis of L. plantarium-loaded microcapsule powder.

### DESCRIPTION OF SPECIFIC EMBODIMENTS OF THE INVENTION

The present invention, in some embodiments thereof, relates to encapsulation and, more particularly, but not exclusively, to microcapsules encapsulating a microorganism, to processes of preparing same and to products and/or formulations such as foods, pharmaceutical, cosmetics and agricultural products and/or formulations containing same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have conceived using microencapsulation as a tool for incorporating living microorganisms into, for example, foods, pharmaceutical, cosmetics and agricultural formulations, to protect the microorganisms, to extend their storage life and to convert them into a powder form for convenient use. Encapsulation of living microorganisms can also promote controlled release and optimized delivery to the site of action, thereby potentiating the efficacy of the respective strain (e.g., a probiotic strain). This process can also prevent microorganisms from multiplying in a formulation that would otherwise change their aesthetic and sensory characteristics.

The selection of the best encapsulation technology for living microorganisms such as probiotics needs to consider numerous aspects to guarantee the survival of the microorganisms during the encapsulation process, in storage conditions and during application, as well as the release mechanism in the specific desired area of application. The most problematic issues encountered in probiotic encapsulation typically include keeping the microorganisms alive during manufacturing and storage and releasing the microorganisms onsite while maintaining their efficacy.

The present inventors have designed and successfully practiced a novel methodology for encapsulation of living microorganisms, while successfully maintaining the viability of the microorganisms during manufacturing and storage. The designed methodology is based on the known solvent-removal method, which utilizes a partially water-miscible organic solvent. The present inventors have surprisingly uncovered that the viability of the microorganisms is maintained in such a solvent, under the designed process conditions (see, FIG. 3). In order to further assure compatibility and viability maintenance of the living microorganism, the process parameters and materials have been adjusted. For example, a fatty substance was included in the formulation in order to protect the microorganisms from hydration during the manufacturing process; and wall-forming materials, plasticizers and all other materials were selected as compatible with the microorganisms.

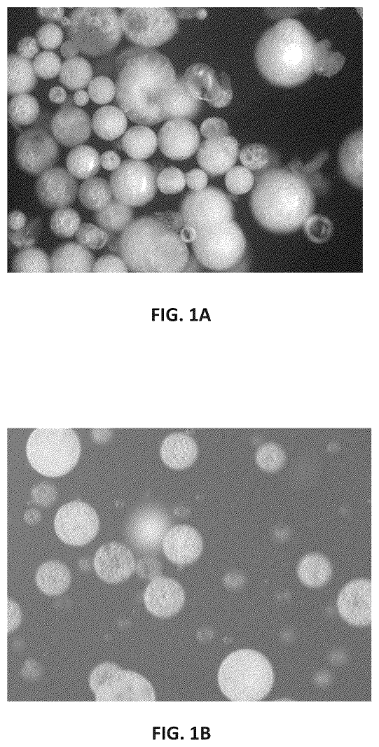

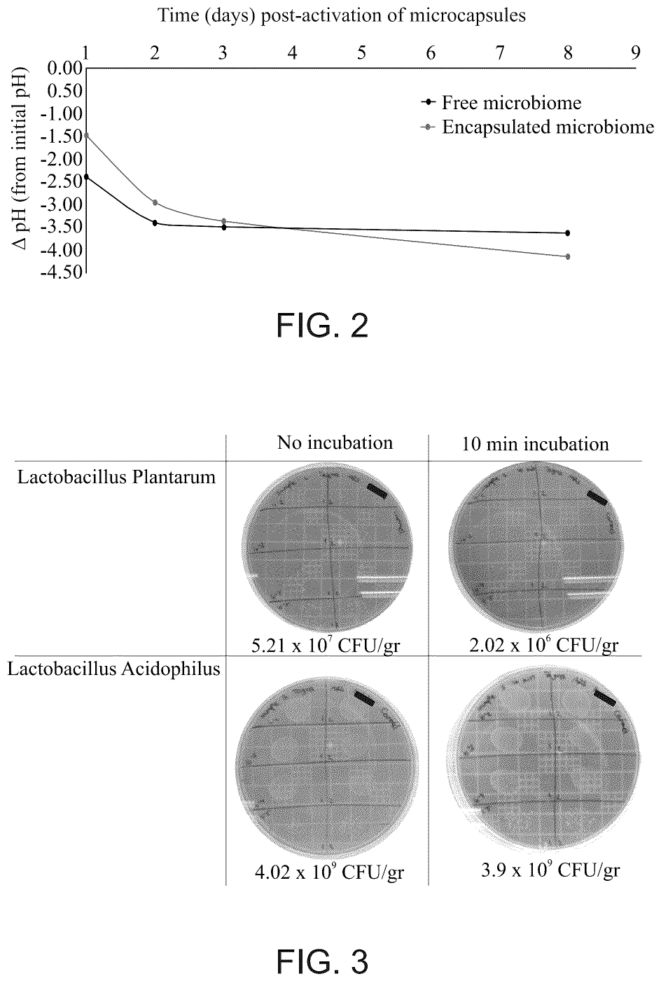

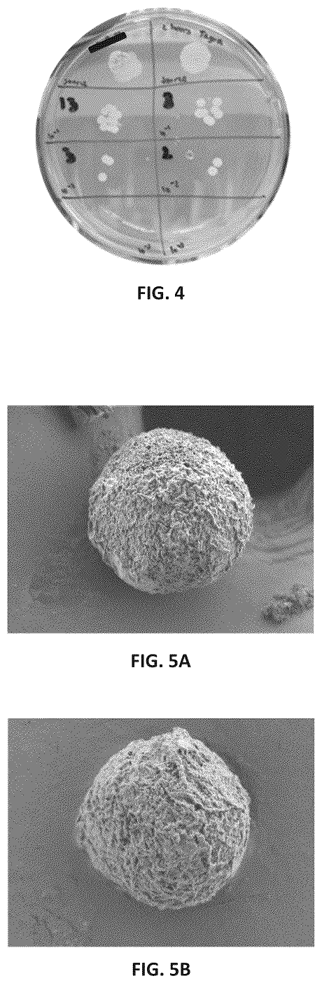

Thus, using a process that is based on known methodologies of encapsulating inanimate substances, the present inventors were able to successfully encapsulate living microorganisms (see, FIGS. 1 and 2), and have demonstrated that the encapsulated microorganisms maintained their viability (see, for example FIG. 3). The encapsulation of the living microorganisms provided adequate protection of microorganisms from environmental conditions (sec, FIGS. 4 and 5A-B).

The physico-chemical properties of the selected wall-forming polymer, and optionally the fatty substance may determine the mechanism of release of the live microorganisms from the obtained microcapsules, and the selection of these components can be made accordingly.

The microcapsules are water-insoluble in order to maintain their structural integrity in the final formulation or product. The final formulation or product can be a cosmetic preparation, a food matrix and the like. The microcapsules can be single-layered, double-layered, triple-layered, and so forth. The microcapsules can be further coated with an additional material to provide a double membrane which can further avoid their exposure to oxygen during storage and can enhance the resistance of the live microorganisms to extreme conditions.

Embodiments of the present invention therefore relate to novel microcapsules, composed of an inner core that comprises living microorganisms and an outer shell made of a selected wall-forming material and optionally a fatty substance and/or a plasticizer, to processes of preparing same, and to products containing same.

### The Microcapsules

The microcapsules provided by the present embodiments are particles (e.g., generally spherical particles), which are generally closed structures containing an encapsulated (enveloped, entrapped) live (living) microorganism. The microcapsules generally have a core-shell structural feature, namely each microcapsule is comprised of a polymeric shell and a core that comprises the live microorganism or may be consisted of the live microorganism, enveloped by the shell.

The shell of the microcapsule is typically applied as a wall-forming material and serves as a membrane for the encapsulated microorganism.

The outer shell may further comprise a plasticizer to control its hardness, and is designed such that the microcapsules are rupturable upon rubbing or pressing on the skin or other animate substrate, typically using a human hand force or equivalent shear force.

In some embodiments, the microcapsules are rupturable upon application of a mechanical pressure. In some embodiments, application of a mechanical pressure comprises a rubbing action (e.g., application of one or more circular motion(s) to microcapsules that contact a surface such as a skin tissue), for example, by a human hand. In some embodiments, at least 20%, or at least 30%, or preferably at least 40% or at least 50%, or at least 60%, or at least 70% or at least 80%, or at least 90% or more, including 100%, of the microcapsules rupture upon being subjected to rubbing, for example, by circle motions (e.g., one or more, or two or more, circle motions) by a human hand.

In some of any of the embodiments described herein, the microcapsules are single-layer microcapsules, comprising a single outer shell enveloping the inner core.

In some other embodiments, the microcapsules are double-layer, or triple-layer, or multi-layer microcapsules, comprising additional one or more layers enveloping the shell layer that envelopes the inner core.

A multi-layer microcapsule is featured as comprising an inner core microcapsule comprising a core which comprises a living microorganism, as described herein, being enveloped by a first shell comprised of a first wall-forming material, and at least one additional shell comprised of a second wall forming material enveloping the first shell, which can be regarded as enveloping a single-layer microcapsule as described herein (comprising the microorganism-containing inner core and a first shell of a first wall-forming material).

Each shell in the multi-layered microcapsules is typically and independently applied as a wall-forming material (e.g., a first, second, third and so forth wall-forming materials forming the first, second, third, and so forth, outer shells, respectively), and serves as a membrane for the encapsulated substance. The wall-forming polymers of the first, second, and optionally third, fourth and so forth, can be the same or different.

The microcapsules of the present embodiments, among other uses, are suitable for inclusion in topical, e.g., cosmetic, cosmeceutical and pharmaceutical (e.g., dermatological), applications. When applied to the skin, the microcapsules are capable of being ruptured upon application of shear forces such as rubbing and pressing on the skin, but they remain intact in the formulation itself before application, and exhibit exceptional stability in water-based, oil-based, silicon-based and emulsion-type formulations. The microcapsules are hard enough to avoid destruction of the shells and realization of the content during production processes such as isolation/filtration, drying, sieving, etc., and/or during storage.

The microcapsules according to the present embodiments are also referred to herein as microorganism-encapsulating microcapsules or as microcapsules encapsulating a live or living microorganism.

In some embodiments, the microcapsules encapsulating the microorganism as described herein are prepared by a solvent removal method, as described hereinunder and exemplified in the Examples section that follows.

In some embodiments, a mean size of the microcapsules as described herein is within a range of from about 10 μm to about 400 μm, or from about 10 μm to about 300 μm, or from about 10 μm to about 200 μm, or from about 10 μm to about 100 μm, or from about 50 μm to about 100 μm, including any intermediate value or subranges therebetween.

By “size” it is meant a size of at least one cross-section of the microcapsules, preferably a diameter of the microcapsules.

Herein throughout, a “mean” diameter means an average size of the microcapsules. The size of the microcapsules may be measured, for example, by a Laser distribution size method and particularly by measuring the values D[50] and D[90].

D50 means the size of which 50% of the microcapsules do not exceed (and 50% of the microcapsules do exceed), and D90 means the size of which 90% of the microcapsules do not exceed (and 10% of the microcapsules do exceed).

In some of any of the embodiments described herein, the outer shell comprises, in addition to the wall-forming material, a fatty substance and/or a plasticizer, as described herein.

According to some of any of the embodiments of the invention, a microcapsule as described herein is rupturable or breakable when applied to the skin; that is, a microcapsule as described herein remains intact in a formulation containing same and during industrial processes, but readily breaks when pressed of rubbed on the skin. The non-breakability of the microcapsules before topical application thereof is routinely assessed by monitoring (e.g., using a light microscope) the ability of the microcapsules in a basic cream or lotion to sustain their size and shape when subjected to low shear mixing at e.g., 40-600 (or 80-100) rpm for 5-10 minutes at room temperature and at 40° C. A change of less than 10% in the microcapsule size is indicative of the non-breakability of the microcapsules upon routine industrial processes.

The microcapsules provided herein have shown exceptional stability under conditions that are known to adversely affect live microorganism, as demonstrated in the Examples section that follows.

### The Inner Core

The inner core in the microcapsules described herein comprises a living microorganism.

The term “microorganism” as used herein and in the art describes an organism that is typically microscopic (too small to be seen by the naked human eye) and/or unicellular. Microorganisms are very diverse and include bacteria, fungi, archaca, and protists; microscopic plants (called green algae); and animals such as plankton, the planarian and the amoeba.

Microorganisms encompass, inter alia, probiotics, prebiotics and post-biotics.

The term “Probiotic” is used herein to refer to an organism, typically a microorganism as defined herein and known in the art, with potential health benefit to a subject. The term “probiotic microorganisms” and “probiotics” encompass, for example, probiotic bacteria, probiotic fungi and probiotic yeast. The probiotic organism according to some embodiments of the present invention is a bacterial strain, a fungal strain or a yeast strain. According to some embodiments, the probiotic organism is a Lactobacillus strain such as Lactobacillus acidophilus or Lactobacillus paracasei, or any other Lactobacillus strain known in the art. The probiotic organism can also be a Bifidobacterium strain. Each possibility represents a separate embodiment of the present invention.

Examples of probiotic microorganisms include, but are not limited to, Lactobacillus curvatus, Micrococcus spp., Saccharomyces sp., Saccharomyces boulardii, Staphylococcus epidermidis, Staphylococcus salivarius, Lactococcus sp. HY449, Enterococcus faecalis, Alcaligenes faecalis, Bacillus subtilis, Bacillus megaterium, Bacillus velezensis, Bacillus okhensis, Bacillus polymyxa, Bacillus subtilis GB03, Bacillus pumilus GB34, Exophiala sp., Fusarium culmorum, Halomonas sp., Novosphingobium sp., Penicillium sp., Penicillium minioluteum, Pseudomonas sp., Pseudomonas fluorescens, Pseudomonas alcaligenes, Pseudomonas chlororaphis, Pseudomonas mendocina, Pseudomonas spp., Pantoea sp., Sinorhizobium meliloti, Trichoderma longibrachiatum, Trichoderma harzianum, Trichoderma harzianum subsp. T22, Enterobacter sp., Enterobacter ludwigii, Arthrobacter sp., Ampelomyces quisqualis, Azospirillum lipoferum, Azospirillum spp., Bradyrhizobium japonicum, Delftia acidovorans, Streptomyces griseoviridis K61, Ralstonia sp., Escherichia fergusonii, Acinetobacter calcoaceticus, Salmonella enterica, Brevibacillus choshinensis, Pectobacterium carotovorum, Microbacterium testaceum, Mycobacterium phlei, Cedecea davisae, Curtobacterium M84, Arthrobacter oxidans BB1, Rhizobium spp., Acetobacter sp., Azotobacter spp., Colletotrichum spp., Agrobacterium tumefaciens, Zhinguelliuella spp., Brachybacterium saurashtrense, Vibrio spp., Brevibacterium casei, Haererohalobacter spp., Azotobacter chroococcum W5, Mesorhizobium ciceri F75, Pseudomonas striata P27, Serratia marcescens L11, Anabaena torulosa, Pseudomonas GGRJ21, Bacillus cereus Pb25, Bacillus amyloliquefaciens RWL-1, Bacillus coagulans GBI-30, 6086, Bacillus subtilis var. natto, Bacillus sp., Bifidobacterium Lafti®™ B94, Bifidobacterium bifidum, Bifidobacterium breve Rosell-70, Bifidobacterium bifidum rosell-71, Bifidobacterium breve, Bifidobacterium infantis, Bifidobacterium lactis, Bifidobacterium longum, Bifidobacterium longum Rose-175, Bifidobacterium longum BB 536, Bifidobacterium animalis, Bifidobacterium animalis subsp. lactis BB-12, Bifidobacterium animalis subsp. lactis HN019, Bifidobacterium infantis 35624, Escherichia coli M-17, Escherichia coli Nissle 1917, Lactobacillus acidophilus, Lactobacillus acidophilus LA5, Lactobacillus acidophilus Lafti®™ L10, Lactobacillus casei F19, Lactobacillus casei L19, Lactobacillus casei 431, Lactobacillus casei Lafti®™ L26, Lactobacillus brevis, Lactobacillus bulgaricus, Lactobacillus casei, Lactobacillus gasseri, Lactobacillus paracasei, Lactobacillus plantarum, Lactobacillus plantarum 299v, Lactobacillus pentosus, Lactobacillus reuteri, Lactobacillus reuteri ATTC 55730 (Lactobacillus reuteri SD2112), Lactobacillus rhamnosus, Lactobacillus salivarius, Lactobacillus delbrueckii, Lactobacillus fermentum, Lactococcus lactis; Lactococcus lactis subsp, Lactococcus lactis Rosell-1058, Lactococcus lactis subsp. lactis L1A, Lactobacillus paracasei St11, Lactobacillus paracasei NCC2461, Lactobacillus fortis, Lactobacillus johnsonii La1 (Lactobacillus johnsonii NCC 533), Lactobacillus rhamnosus Rosell-11, Lactobacillus rhamnosus LGG, Lactobacillus rhamnosus LB21, Lactobacillus rhamnosus 271, Lactobacillus acidophilus Rosell-52, Streptococcus thermophilus, Streptococcus sp., Diacetylactis spp., Saccharomyces cerevisiae, Enterococcus spp., Pediococcus spp., Propionibacterium spp., and Peptosteptococcus spp.

The term “living microorganism” as used herein and in the art describes a microorganism, as described herein, that exhibits metabolic functions and/or is successfully qualifies as a viable microorganism by at least one technique known to determine viability and/or activity.

Examples of viability assessment techniques (live/dead determination) include cultivation, membrane integrity, isothermal microcalorimetry, proteomics, Bioorthogonal noncanonical amino acid tagging (BONCAT), RNA-based methods, viability PCR, isotope labelling, and are described in Emerson et al. Microbiome (2017) 5:86.

As used herein, the term “CFU” means “colony forming unit” and is a measure of viable cells in which a colony represents an aggregate of cells derived from a single progenitor cell.

The terms “microbiome” or “microbiota” are used interchangeably, and refer to collectively, to the entirety of microbes found in association with a higher organism, such as a human. Organisms belonging to a human's microbiota may generally be categorized as bacteria, archaca, yeasts, and single-celled eukaryotes, as wells as various parasites such as Helminths.

The term “probiotic” utilizes the World Health Organization's 2001 definition of “live micro-organisms which, when administered in adequate amounts, confer a health benefit on the host”. Probiotics must be alive when administered, have viability and reproducibility based on in vivo results, and during use and storage.

According to some of any of the embodiments described herein, the living microorganism is a bacterium.

According to some embodiments of the present invention, the living microorganism is a lactic acid bacterium.

In some embodiments, the living microorganism can be a lactic acid bacterium. “Lactic acid bacterium” refers to a Glade of Gram positive, low-GC, acid tolerant, non-sporulating, non-respiring rod or cocci that are associated by their common metabolic and physiological characteristics. These bacteria, usually found in decomposing plants and lactic products produce lactic acid as the major metabolic end product of carbohydrate fermentation. The lactic acid bacterium can be, for example, of the genera Lactobacillus, Leuconostoc, Pediococcus, Lactococcus, and Streptococcus.

According to some embodiments of the present invention, the living microorganism is a probiotic microorganism.

The microorganisms of the present invention can be selected according to an intended use of a product comprising the microcapsules.

Exemplary probiotic microorganisms that are beneficial for use in a pharmaceutical product include but are not limited to, Bacillus coagulans GBI-30, 6086, Bacillus subtilis var natt, Bacillus sp., Bifidobacterium Lafti®™ B94, Bifidobacterium bifidum, Bifidobacterium bifidum rosell-71, Bifidobacterium breve, Bifidobacterium breve Rosell-70, Bifidobacterium infantis, Bifidobacterium lactis, Bifidobacterium longum, Bifidobacterium longum Rose-175, Bifidobacterium animalis, Bifidobacterium animalis subsp. lactis BB-12, Bifidobacterium animalis subsp. lactis HN019, Bifidobacterium infantis 35624, Escherichia coli M-17, Escherichia coli Nissle 1917, Lactobacillus acidophilus, Lactobacillus acidophilus Lafti®™ L10, Lactobacillus casei Lafti®™ L26, Lactobacillus brevis, Lactobacillus bulgaricus, Lactobacillus casei, Lactobacillus gasseri, Lactobacillus paracasei, Lactobacillus plantarum, Lactobacillus pentosus, Lactobacillus reuteri ATTC 55730 (Lactobacillus reuteri SD2112), Lactobacillus rhamnosus, Lactobacillus salivarius, Lactobacillus delbrueckii, Lactobacillus fermentum, Lactococcus lactis; Lactococcus lactis subsp, Lactococcus lactis Rosell-1058, Lactobacillus paracasei St11, Lactobacillus paracasei NCC2461, Lactobacillus fortis, Lactobacillus johnsonii La1, Lactobacillus rhamnosus Rosell-11, Lactobacillus acidophilus Rosell-52, Streptococcus thermophilus, Streptococcus sp., Diacetylactis spp., Saccharomyces cerevisiae, Enterococcus spp., Pediococcus spp., Propionibacterium spp., Peptosteptococcus spp., and any combination or (e.g., genetically-modified) variants thereof.

Exemplary probiotic microorganisms that are beneficial for use in a cosmetic product include but are not limited to, Bifidobacterium spp., Lactobacillus sp., Lactobacillus curvatus, Lactobacillus paracasei, Lactobacillus pentosus, Lactobacillus plantarum, Bacillus spp., Streptococcus spp., Micrococcus spp., Saccharomyces sp., Saccharomyces boulardii, Staphylococcus epidermidis, Staphylococcus salivarius, Lactococcus sp. HY449, Streptococcus thermophiles, Enterococcus faecalis, and any combination or (e.g., genetically-modified) variants thereof.

Exemplary probiotic microorganisms that are beneficial for use in a cosmeceutical product include but are not limited to, Staphylococcus epidermidis, Staphylococcus salivarius, Lactococcus sp. HY449, Streptococcus thermophiles, Enterococcus faccalis, Lactobacillus plantarum, Lactobacillus pentosus, and any combination or (e.g., genetically-modified) variants thereof.

Exemplary probiotic microorganisms that are beneficial for use in a food or any other edible product include, but are not limited to, Bacillus coagulans GBI-30, 6086, Bacillus subtilis var. natto, Bifidobacterium Lafti®™ B94, Bifidobacterium bifidum, Bifidobacterium bifidum rosell-71, Bifidobacterium breve, Bifidobacterium breve Rosell-70, Bifidobacterium infantis, Bifidobacterium lactis, Bifidobacterium longum, Bifidobacterium longum Rose-175, Bifidobacterium longum BB 536, Bifidobacterium animalis, Bifidobacterium animalis subsp. animalis, Bifidobacterium animalis subsp. lactis BB-12, Bifidobacterium animalis subsp. lactis HN019, Bifidobacterium infantis 35624, Escherichia coli M-17, Escherichia coli Nissle 1917, Lactobacillus acidophilus, Lactobacillus acidophilus LA5, Lactobacillus acidophilus Rosell-52, Lactobacillus acidophilus Lafti®™ L10, Lactobacillus casei, Lactobacillus casei Lafti®™ L26, Lactobacillus casei L19, Lactobacillus casei F19, Lactobacillus casei 431, Lactobacillus brevis, Lactobacillus bulgaricus, Lactobacillus gasseri, Lactobacillus paracasei, Lactobacillus plantarum, Lactobacillus plantarum 299v, Lactobacillus reuteri, Lactobacillus reuteri ATTC 55730, Lactobacillus rhamnosus, Lactobacillus rhamnosus Rosell-11, Lactobacillus rhamnosus LGG, Lactobacillus rhamnosus LB21, Lactobacillus rhamnosus 271, Lactobacillus salivarius, Lactobacillus delbrueckii, Lactobacillus fermentum, Lactococcus lactis, Lactococcus lactis subsp. lactis L1A, Lactococcus lactis Rosell-1058, Lactobacillus paracasei St11, Lactobacillus paracasei NCC2461, Lactobacillus fortis, Lactobacillus johnsonii, Lactobacillus johnsonii La1, Lactobacillus pentosus, Streptococcus thermophilus, Diacetylactis, Saccharomyces cerevisiae, and any combination or (e.g., genetically-modified) variants thereof.

Exemplary probiotic microorganisms that are beneficial for use in an agricultural product include, but are not limited to, Alcaligenes faecalis, Bacillus sp., Bacillus subtilis, Bacillus megaterium, Bacillus velezensis, Bacillus okhensis, Bacillus polymyxa, Bacillus subtilis, Exophiala sp., Fusarium culmorum, Halomonas sp., Lactobacillus plantarum, Novosphingobium sp., Penicillium minioluteum, Penicillium sp., Phoma glomerata, Pseudomonas sp., Pseudomonas fluorescens, Pseudomonas alcaligenes, Pseudomonas chlororaphis, Pseudomonas mendocina, Pseudomonas spp., Pantoca sp., Sinorhizobium meliloti, Trichoderma longibrachiatum, Trichoderma harzianum, Trichoderma harzianum subsp. T22, Enterobacter sp., Arthrobacter sp., Enterobacter ludwigii, Lactobacillus plantarum, Ampelomyces quisqualis M-10, Azospirillum lipoferum, Azospirillum spp., Bacillus subtilis GB03, Bradyrhizobium japonicum, Bacillus pumilus GB34, Coniothyrium minitans, Delftia acidovorans, Phlebiopsis gigantean, Streptomyces griscoviridis K61, Ralstonia sp., Escherichia fergusonii, Acinetobacter calcoaceticus, Salmonella enterica, Brevibacillus choshinensis, Pectobacterium carotovorum, Microbacterium testaceum, Mycobacterium phlei, Cedecea davisae, Curtobacterium M84, Arthrobacter oxidans BB1, Rhizobium spp., Acetobacter spp., Azotobacter spp., Colletotrichum spp., Agrobacterium tumefaciens, Zhinguelliuella spp., Brachybacterium saurashtrense, Vibrio spp., Brevibacterium casei, Haererohalobacter spp., Azotobacter chroococcum W5, Mesorhizobium ciceri F75, Pseudomonas striata P27, Serratia marcescens L11, Anabaena torulosa, Pseudomonas GGRJ21, Bacillus cereus Pb25, Bacillus amyloliquefaciens RWL-1, and any combination or (e.g., genetically-modified) variants thereof.

The inner core may comprise one or more type(s) of the living microorganisms, as long as they provide a mutual activity and their viability and activity is maintained. In order words, when two or more types of microorganisms are included in an inner core, they should be compatible with one another, as defined herein. A person skilled in the art will know how to choose combinations of microorganisms to produce a desired effect.

According to some embodiments of the present invention, the amount of the living microorganism, which constitute the inner core of the microcapsules, is within a range of from about 1% to about 90%, or from about 1% to about 50%, or from about 1% to about 30%, or from about 5% to about 50%, or from about 5% to about 25%, or from about 10% to about 50%, or from about 10% to about 30%, by weight, or from about 10% to about 20%, by weight of the total weight of the microcapsule, including any subranges and any intermediate values therebetween.

### The Wall-Forming Material

The wall-forming material forms the outer shell(s) of the microcapsules of the present embodiments, and serves as a membrane for the encapsulated substance (the living microorganism.). According to embodiments of the present invention, the wall forming material forming the outer shell(s) comprises a wall-forming polymer or co-polymer.

The phrase “wall-forming polymer”, which is also referred to herein as “wall-forming polymeric material” refers to a polymeric material (e.g., a polymer or copolymer) or a combination of two or more different polymeric materials, as defined herein, which form a component of the external wall or layer or shell of single-layer microcapsules, or, in the case of multi-layer microcapsules, additionally of the one or more intermediate shells between the inner core and the external (outer most) layer. In the context of single-layer microcapsules, the term “polymer shell” refers to a polymer layer comprised of the wall-forming polymer(s), which envelopes the inner core. In the context of multi-layer microcapsules, the term “polymer shell” refers to any of the polymer layers which envelopes the inner core, or which envelopes the preceding polymer layer.

In some embodiments, the wall-forming polymer is selected so as to sustain shear forces applied while being compounded in industrial processes. In some embodiments, the wall-forming polymer is selected so as to sustain shear forces applied while being compounded in industrial processes, but, nevertheless, so as to provide microcapsule which are rupturable when applied (e.g., rubbed or pressed) on the skin.

The amount (weight/weight) of the wall-forming polymeric material(s) of the outer shell relative to the total microcapsule weight can be within a range of from about 5% to about 95%, or from about 10% to about 95%, or from about 10% to about 80%, or from about 20% to about 80%, or from about 10% to about 50%, or from about 20% to about 50%, by weight, including any subranges and any intermediate values therebetween.

In embodiments related to multi-layer microcapsules, the wall-forming material in each of the outer shells in the microcapsules described herein (e.g., a first wall-forming material of the inner core, a second wall-forming material of a first outer shell enveloping the inner core, and optionally a third wall-forming material of a second outer shell enveloping the first outer shell, and so forth) can be the same or different.

According to some of any of the embodiments described herein, the wall-forming polymer is characterized as having a positive Log P value.

The term “Log P” refers to the logarithm to the base 10 of P, the partition coefficient. The degree of hydrophobicity of an organic compound can be correlated with its octanol/water partition coefficient P. The octanol/water partition coefficient of a compound is the ratio between its equilibrium concentration in octanol and in water. An organic compound with a greater partition coefficient P is considered to be more hydrophobic, less miscible with aqueous solutions and more miscible with organic solvents. Conversely, an organic compound with a smaller partition coefficient P is considered to be more hydrophilic or more miscible with aqueous solutions. Since the partition coefficients of organic compounds normally have high values, they are more conveniently given in the form of their logarithm to the base 10, Log P.

According to some of any of the embodiments described herein, the wall-forming polymeric material has a log P value in a range of from 2 to 20, or from 2 to 10, or from 4 to 10, including any intermediate values and subranges therebetween.

According to some of any of the embodiments described herein, the wall-forming polymeric material is dissolvable at a concentration of at least 100 grams per liter (at least 10% by weight) in a partially water-miscible organic solvent that is compatible with a living microorganism (e.g., a selected microorganism in the inner core).

By “dissolvable”, as used herein, it is meant that at least 10%, or at least 20%, preferably at least 50%, or at least 60%, preferably at least 80%, or at least 90%, of the indicated material, when contacting a solution (e.g., an organic solvent as described herein), dissolves. In some embodiments, at least 50% or at least 80% of the indicated material dissolves upon contacting the (e.g., organic) solution, during a time period that ranges from a few minutes to a few hours, depending on the temperature and other conditions of the contacting and on the size and shape of the formed hardened material. In some embodiments, at least 50% or at least 80% of the indicated material dissolves upon contacting the (e.g., organic) solution, during a time period of 8 hours at room temperature and atmospheric pressure.

According to some of any of the embodiments described herein, the wall-forming polymeric material is dissolvable, as defined herein, at a concentration of from about 100 grams per liter (at least 10% by weight) to about 1000 grams per liter (100%), or from about 200 grams per liter (at least 20% by weight) to about 1000 grams per liter (100%), from about 300 grams per liter (at least 10% by weight) to about 1000 grams per liter (100%), or from about 400 grams per liter (at least 40% by weight) to about 1000 grams per liter (100%), or from about 500 grams per liter (at least 10% by weight) to about 1000 grams per liter (100%), or from about 100 grams per liter (at least 10% by weight) to about 800 grams per liter (80%), or from about 500 grams per liter (at least 50% by weight) to about 1000 grams per liter (100%), or from about 500 grams per liter (at least 10% by weight) to about 800 grams per liter (80%), including any intermediate values and subranges therebetween, in a partially water-miscible organic solvent that is compatible with a living microorganism (e.g., a selected microorganism in the inner core), as defined herein.

The phrase “water-miscible”, as used herein, refers to organic solvents that are soluble and/or dispersible in water (e.g., when mixed at room temperature at equal volumes, that is, having solubility in water of at least 50% by volume). Several factors inherent in the structure of the solvent molecules can affect the miscibility of organic solvents in water, such as for example, the length of the carbon chain and the type of functional groups therein.

The phrase “partially water-miscible organic solvent” describes an organic solvent that is soluble in water only at less than 50% at room temperature, for example, at a volume concentration of from about 10 to about 50%, by volume.

Exemplary such solvents include ethyl acetate, alcohols such as ethanol, and ethyl formate.

The term “biocompatible” generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when contacted with an animate subject.

The phrase “compatible with the microorganism” describes a material or solvent that when contacted with a selected microorganism do not cause cell death or any other adverse effect to the microorganism. A material or solvent that is compatible with a microorganism is such that when contacted with the microorganism does not affect is viability. Methods of determining compatibility with a microorganism are well known in the art and some are exemplified in the Examples section that follows.

Compatibility of a solvent with a living microorganism can be determined using methods well-known in the art for determining a microorganism viability. An exemplary such assay is provided in the Examples section that follows.

According to some of any of the embodiments described herein, a partially water-miscible organic solvent is or comprises ethyl acetate and the wall-forming polymeric material is dissolvable in ethyl acetate at a concentration of at least 100 grams per liter, or from 100 grams to 1000 grams per liter, or from 500 grams to 1000 grams per liter, as described herein in any of the respective embodiments, including any intermediate values and subranges therebetween.

According to some of any of the embodiments described herein, the wall-forming polymer is characterized as having a positive Log P value as described herein in any of the respective embodiments and is dissolvable at a concentration of at least 100, or at least 200, or at least 300, or at least 400, or at least 500, grams per liter in a partially water-miscible organic solvent that is compatible with a living microorganism, as described and defined herein (e.g., ethyl acetate).

According to some of any of the embodiments described herein, the wall-forming polymer comprises a biocompatible polymer.

According to some of any of the embodiments described herein, the wall-forming polymer is selected as compatible, as defined herein, with the encapsulated microorganism.

Compatibility of a wall-forming agent with a living microorganism can be determined using methods well-known in the art for determining a microorganism viability.

According to some of any of the embodiments described herein, the wall-forming polymer comprises a naturally occurring polymer, that is, the wall-forming polymer can be found or can be extracted from a natural source (e.g., plants, minerals, etc.). A naturally occurring polymer can be extracted from a natural source or can be synthetically prepared.

According to some of any of the embodiments described herein, the wall-forming material is a naturally-occurring polymer, as defined herein, and is compatible with the microorganism, as defined herein.

According to some of any of the embodiments described herein, the wall-forming material is a naturally-occurring polymer, as defined herein, is compatible with the microorganism, as defined herein, and has a positive Log P as defined herein.

According to some of any of the embodiments described herein, the wall-forming material is a naturally-occurring polymer, as defined herein, is compatible with the microorganism, as defined herein, and is dissolvable in a partially water-miscible organic solvent that is compatible with the microorganism, as defined herein.

According to some of any of the embodiments described herein, the wall-forming material is a naturally-occurring polymer, as defined herein, is compatible with the microorganism, as defined herein, has a positive Log P as defined herein, and is dissolvable in a partially water-miscible organic solvent that is compatible with the microorganism, as defined herein.

In some embodiments, one or more, or each, of the wall-forming polymeric materials forming the outer shell(s) comprises a polyacrylate, a polymethacrylate, a cellulose ether or ester, or any combination thereof.

Exemplary wall-forming polymeric materials that are usable in the context of the present embodiments include, but are not limited to, polyacrylates, polymethacrylates, low molecular weight poly(methyl methacrylate)-co-(methacrylic acid) (e.g., 1:0.16), poly(ethyl acrylate)-co-(methyl methacrylate)-co-(trimethylammmonium-ethyl methacrylate chloride) (e.g., 1:2:0.1) (also known as Eudragit® RSPO), poly(butyl methacrylate)-co-(2-dimethylaminoethyl methacrylate)-co-(methyl methacrylate) (e.g., 1:2:1), poly(styrene)-co-(maleic anhydride), copolymer of octylacrylamide, cellulose ethers, cellulose esters, poly(ethylene glycol)-block-poly(propylene glycol)-block-poly(ethylene glycol), PLA (poly(lactic acid), PGA (poly(glycolide), PLGA (poly(lactide)-co-poly(glycolide) or any combination thereof.

Any combination of polymers and co-polymers as described herein is contemplated for a wall-forming material, as described herein.

In some embodiments, the wall-forming polymeric material of an outer shell comprises a cellulose ether or cellulose ester such as, but not limited to, methyl cellulose, ethyl cellulose, hydroxypropyl methyl cellulose, hydroxypropyl cellulose, cellulose acetate, cellulose acetate phthalate, cellulose acetate butyrate and hydroxypropyl methyl cellulose acetate phthalate. When a cellulose ether or ester is used in the polymeric material, it preferably contains about 1-20, or 1-10, or 1-5, % hydroxyl groups which are free to form hydrogen bonds (e.g., hydroxyl groups which are not alkylated or acylated).

In some of any of the embodiments described herein, the wall forming material is or comprises a cellulose ester.

In some of any of the embodiments described herein, the cellulose ester comprises no more than 10% or no more than 5%, or no more than 4%, or no more than 3%, free hydroxy group. In some of any of the embodiments described herein, the % (content) of the esterified groups in the cellulose ester ranges from 10 to 60, or from 10 to 50, or from 10 to 40, %.

In some of any of the embodiments described herein, the cellulose ester is cellulose acetate.

In some of any of the embodiments described herein, the cellulose ester is cellulose acetate butyrate.

According to some of any of the embodiments described herein, an average molecular weight (Mn) of a wall-forming polymer as described herein (e.g., a cellulose ester) ranges from 1,000 to 100,000 grams/mol, or from 1,000 to 50,000, or from 1,000 to 30,000, or from 10,000 to 100,000, or from 10,000 to 50,000, or from 10,000 to 40,000, or from 5,000 to 50,000, or from 5,000 to 40,000, or from 5,000 to 30,000, or from 10,000 to 30,000, grams/mol, including any intermediate values and subranges therebetween.

Depending on the intended use, the wall-forming material as be selected as biodegradable. Such wall-forming materials are usable in, for example, microcapsules intended to be included in pharmaceutical products for administration to the body.

The term “biodegradable” generally refers to a polymer that will degrade or erode by enzymatic action and/or hydrolysis under physiologic conditions to smaller units or chemical species that are capable of being metabolized, eliminated, or excreted by the subject. The degradation time is a function of polymer composition, morphology, such as porosity, particle dimensions, and environment.

Biodegradable wall-forming polymers are suitable for use in, for example, pharmaceutical products, agricultural products, and food products, as described herein, and also in some cosmetic or cosmeceutical products as described herein.

### The Fatty Substance

The phrases “oily substance” and “fatty substance” are used herein interchangeably.

According to some of any of the embodiments described herein, the outer shell further comprises a fatty substance.

The term “fatty substance” describes a water-immiscible non-aqueous substance which can be solid or liquid at room temperature (25° C.) and atmospheric pressure (760 mmHg).

Exemplary fatty substances include, but are not limited to, waxes, lipids, fatty acids, fatty esters, fatty alcohols, plant (vegetable) oils, mineral oils, and certain types of triglycerides.

According to some of any of the embodiments described herein, the fatty substance is a biocompatible fatty substance, as defined herein.

According to some of any of the embodiments described herein, the fatty substance is a naturally occurring fatty substance, that is, the fatty substance can be found in or can be extracted from a natural source (e.g., plants, minerals, etc.). A naturally occurring fatty substance can be extracted from a natural source or can be synthetically prepared.

According to some of any of the embodiments described herein, the fatty substance is a naturally-occurring fatty substance, as defined herein, and is compatible with the microorganism, as defined herein.

According to some of any of the embodiments described herein, the fatty substance is dissolvable in the partially water-miscible organic solvent as described herein.

In some of the embodiments described herein, the fatty substance is dissolvable in the partially water-miscible organic solvent as described herein (e.g., ethyl acetate) at a concentration of at least 100 grams per liter, or at least 200, 300, 400 or 500 grams per liter, or from 100 grams to 1,000 grams per liter, or from 200 grams to 1,000 grams per liter, or from 300 grams to 1,000 grams per liter, or from 400 grams to 1,000 grams per liter, or from 500 grams to 1,000 grams per liter, or from 100 to 800 grams per liter, including any intermediate values and subranges therebetween.

According to some of any of the embodiments described herein, the fatty substance is solid at room temperature and ambient pressure. According to some of these embodiments, the fatty substance is dissolvable in the partially water-miscible organic solvent as described herein and/or is a shear-thinning or thixotropic substance, which becomes more viscous under shear forces, for example, the process parameters as described herein.

According to some of any of the embodiments described herein, the fatty substance is a naturally occurring substance as defined herein and is dissolvable in the partially water-miscible organic solvent as described herein. According to some of these embodiments, the fatty substance is dissolvable in the partially water-miscible organic solvent as described herein and/or is a shear-thinning or thixotropic substance, as described herein.

According to some of any of the embodiments described herein, the fatty substance is a naturally occurring substance as defined herein and is compatible with the microorganism. According to some of these embodiments, the fatty substance is dissolvable in the partially water-miscible organic solvent as described herein and/or is a shear-thinning or thixotropic substance, as described herein.

According to some of any of the embodiments described herein, the fatty substance is a naturally occurring substance as defined herein, is dissolvable in the partially water-miscible organic solvent as described herein and is compatible with the microorganism. According to some of these embodiments, the fatty substance is dissolvable in the partially water-miscible organic solvent as described herein and/or is a shear-thinning or thixotropic substance, as described herein.

According to some of any of the embodiments described herein, the fatty substance is a wax, a lipid and/or an oil, including mineral oils and plant (vegetable) oils.

Exemplary mineral oils include, without limitation, silicon oil, white oil, white mineral oil, liquid petrolatum, liquid paraffin or white paraffin oil. The mineral oil may optionally comprise a mineral oil replacement. Mineral oil replacements include alkanes having at least 10 carbon atoms (e.g., isohexadecane), benzoate esters, aliphatic esters, noncomodogenic esters, volatile silicone compounds (e.g., cyclomethicone), and volatile silicone substitutes. Examples of benzoate esters include C₁₂C₁₅ alkyl benzoate, isostearyl benzoate, 2-ethyl hexyl benzoate, dipropylene glycol benzoate, octyldodecyl benzoate, stearyl benzoate, and behenyl benzoate. Examples of aliphatic esters include C₁₂C₁₅ alkyl octonoate and dioctyl maleate. Examples of noncomodogenic esters include isononyl isononanoate, isodecyl isononanoate, diisostearyl dimer dilinoleate, arachidyl propionate, and isotridecyl isononanoate. Examples of volatile silicone substitutes include isohexyl decanoate, octyl isononanoate, isononyl octanoate, and diethylene glycol dioctanoate.

Exemplary vegetable oils include, but are not limited to, olive oil, canola oil, coconut oil, rapeseed oil, corn oil, cottonseed oil, peanut oil, saffron oil, safflower oil, sesame oil, soybean oil, palm oil, sunflower oil and any combination thereof.

Additional exemplary vegetable oils include nut oils, including, but not limited to, Almond oil, Beech nut oil, Brazil nut oil, Cashew oil, Hazelnut oil, Macadamia oil, Mongongo nut oil (or manketti oil), Pecan oil, Pine nut oil, Pistachio oil, Walnut oil, and Pumpkin seed oil.

Additional exemplary vegetable oils include citrus oils, including, but not limited to, grapefruit seed oil, lemon oil and orange oil.

Additional exemplary vegetable oils are derived from melon and gourd seeds and include, but are not limited to, Bitter gourd oil, from the seeds of Momordica charantia, Bottle gourd oil, extracted from the seeds of the Lagenaria siceraria, Buffalo gourd oil, from the seeds of the Cucurbita foetidissima, Butternut squash seed oil, from the seeds of Cucurbita moschata, Egusi seed oil, from the seeds of Cucumeropsis mannii naudin, Pumpkin seed oil, and Watermelon seed oil, pressed from the seeds of Citrullus vulgaris.

Additional exemplary vegetable oils include, but are not limited to, Açaí oil, Black seed oil, Blackcurrant seed oil, Borage seed oil, Evening primrose oil, Flaxseed oil (called also linseed oil), Amaranth oil, Apricot oil, Apple seed oil, Argan oil, Avocado oil, Babassu oil, Ben oil, Borneo tallow nut oil, Cape chestnut oil, Carob pod oil (Algaroba oil), Cocoa butter, Cocklebur oil, Cohune oil, Coriander seed oil, Date seed oil, Dika oil, False flax oil, Grape seed oil, Hemp oil, Kapok seed oil, Kenaf seed oil, Lallemantia oil, Mafura oil, Marula oil, Meadowfoam seed oil, Mustard oil Niger seed oil, Nutmeg butter, Okra seed oil, Papaya seed oil, Perilla seed oil, Persimmon seed oil, Pequi oil, Pili nut oil, Pomegranate seed oil, Poppyseed oil, Pracaxi oil, Prune kernel oil, Quinoa oil, Ramtil oil, Rice bran oil, Royle oil, Sacha inchi oil, Sapote oil, Seje oil, Shea butter, Taramira oil, Tea seed oil (Camellia oil), Thistle oil, Tigernut oil (or nut-sedge oil), Tobacco seed oil, Tomato seed oil, and Wheat germ oil.

As used herein, the term “lipid” describes a hydrocarbon residue having 3-30 carbon atoms. In naturally-occurring compounds, the lipids in phospholipids and glycerolipids are derived from fatty acids and are therefore attached to the backbone via an O-acyl (ester) bond. The lipid moiety can be attached to the backbone either via and ether or an ester bond.

As used herein, the terms “mono-esterified” and “di-esterified” with respect to phospholipids or glycerolipids, describe phospholipids or glycerolipids, either oxidized or non-oxidized, in which one or two of the lipid moieties, respectively, are attached to the glycerol backbone via an ester (e.g., O-fatty acyl) bond.

As used herein, the terms “mono-etherified” and “di-etherified” with respect to phospholipids or glycerolipids, describe phospholipids or glycerolipids, either oxidized or non-oxidized, in which one or two of the lipid moieties, respectively, are attached to the glycerol backbone via an ether bond.

The term “phosphoglycerol” describes a compound having a glycerolic backbone and a phosphate group attached to one position thereof.

The term “phosphoglycerides” describes a compound having a glycerolic backbone, one or two lipid moieties and a phosphate moiety attached thereto.

The term “mono-etherified phosphoglyceride” describes a phosphoglyceride, in which a lipid moiety is attached to the glycerolic backbone via an ether bond.

By “wax” it is generally meant herein a hydrophobic material, which is solid at room temperature. Waxes can be derived from minerals or plants, or be synthetically prepared. Waxes typically comprise one or more compounds which are each independently a saturated or unsaturated hydrocarbon chain of at least 20 preferably at least 30, or at least 40 carbon atoms in length.

In some embodiments, the hydrocarbon consists of carbon and hydrogen atoms. In some embodiments, the hydrocarbon is 30, 32, 34, 36, 38, 40, or more carbon atoms in length.

By “Paraffin wax” it is meant herein a mineral wax, which is typically obtained by freezing or solvent dewaxing of petroleum fractions, and then deoiling and refining. Paraffin wax typically consists mainly of long alkylene chain(s), e.g., having at least 20, preferably at least 30, or at least 40 carbon atoms in length, which can be saturated and/or unsaturated, linear and/or branched, typically a mixture thereof.

By “vegetable wax” it is meant herein a natural wax material that is derived from one or more plants. “Vegetable wax” is also referred to herein as “natural wax” and encompasses wax substances that are obtainable from plants, or mixtures thereof with other wax substances, and/or synthetic analogs thereof.

Examples of mineral waxes include but are not limited montan wax, peat wax and petroleum wax such as petrolatum, paraffin wax, ozokerite and ceresin wax.

Examples of natural waxes include but are not limited to animal waxes such as beeswax, and plant waxes such as soy wax, carnauba wax, jojoba wax, candelilla wax, rice-bran wax, sugar cane wax and bayberry wax.

According to some of any of the embodiments described herein, the fatty substance is a vegetable (natural) wax.

According to some of any of the embodiments described herein, the vegetable wax is compatible with the microorganism. According to some of these embodiments, the vegetable wax is dissolvable in the partially water-miscible organic solvent as described herein and/or is a shear-thinning or thixotropic substance, as described herein.

According to some of any of the embodiments described herein, the vegetable (natural) wax is or comprises soy wax.

According to some of any of the embodiments described herein, the fatty substance is or comprises soy wax.

According to some of any of the embodiments described herein, an amount of the fatty substance ranges from about 1 to about 20, or from about 1 to about 10, or from about 5 to about 15, % by weight of the total weight of the microcapsule, including any intermediate values and subranges therebetween.

### A Plasticizer

In some embodiments of any of the embodiments of the present invention, an outer shell of the microcapsules further comprises a plasticizer.

Herein and in the art, a “plasticizer” describes a substance which increases the plasticity or fluidity of a composition. In the context of the present embodiments, a plasticizer is added to the wall-forming material in order to control the physical properties and level of elasticity of the microcapsule's outer shells.

According to some of any of the embodiments described herein, the plasticizer is a biocompatible plasticizer as defined herein, and in some embodiments, it is compatible with the microorganism, as defined herein.

Exemplary plasticizers include, but are not limited to, triethyl citrate, tricaprylin, trilaurin, tripalmitin, triacetin, acetyltriethyl citrate, paraffin oil, and any combination thereof. In exemplary embodiments, the plasticizer is tricaprylin (caprylic triglyceride).

The amount of the plasticizer can be within a range of from about 0.5% to about 70%, or from about 5% to about 70%, or from about 10% to about 70%, or from about 20% to about 70%, or from about 30% to about 70%, or is about 50% by weight, of the total weight of the microcapsule, including any subranges and any intermediate values therebetween.

### Exemplary Microcapsules

According to some embodiments of the present invention, the microcapsule comprises an inner core enveloped by an outer shell formed of a wall-forming polymeric material, the inner core comprises a living microorganism, as described herein in any of the respective embodiments, and the microcapsule is characterized by:
(a) a wall-forming polymeric material having a positive log P value, as described herein in any of the respective embodiments; and/or
(b) the microcapsule is rupturable by a mechanical action of rubbing or pressing with a human hand, as described herein in any of the respective embodiments; and/or
(c) the wall-forming polymeric material is dissolvable at a concentration of at least 100 grams per liter in a partially water-miscible organic solvent that is compatible with the microorganism, as described herein in any of the respective embodiments.

According to some embodiments of the present invention, the microcapsule comprises an inner core enveloped by an outer shell formed of a wall-forming polymeric material, the inner core comprises a living microorganism, as described herein in any of the respective embodiments, and the microcapsule is characterized by at least (a).

According to some embodiments of the present invention, the microcapsule comprises an inner core enveloped by an outer shell formed of a wall-forming polymeric material, the inner core comprises a living microorganism, as described herein in any of the respective embodiments, and the microcapsule is characterized by at least (b).

According to some embodiments of the present invention, the microcapsule comprises an inner core enveloped by an outer shell formed of a wall-forming polymeric material, the inner core comprises a living microorganism, as described herein in any of the respective embodiments, and the microcapsule is characterized by at least (c).

According to some embodiments of the present invention, the microcapsule comprises an inner core enveloped by an outer shell formed of a wall-forming polymeric material, the inner core comprises a living microorganism, as described herein in any of the respective embodiments, and the microcapsule is characterized by at least (a) and (b).

According to some embodiments of the present invention, the microcapsule comprises an inner core enveloped by an outer shell formed of a wall-forming polymeric material, the inner core comprises a living microorganism, as described herein in any of the respective embodiments, and the microcapsule is characterized by at least (a) and (c).

According to some embodiments of the present invention, the microcapsule comprises an inner core enveloped by an outer shell formed of a wall-forming polymeric material, the inner core comprises a living microorganism, as described herein in any of the respective embodiments, and the microcapsule is characterized by at least (b) and (c).

According to some embodiments of the present invention, the microcapsule comprises an inner core enveloped by an outer shell formed of a wall-forming polymeric material, the inner core comprises a living microorganism, as described herein in any of the respective embodiments, and the microcapsule is characterized by at least (a) and (b) and (c).

According to some of these embodiments, the outer layer further comprises a fatty substance and/or a plasticizer, as described herein in any of the respective embodiments

According to some of any of the embodiments described herein, the wall-forming material, and the fatty substance and/or plasticizer if present, are all selected biocompatible, compatible with the microorganism and/or as naturally occurring substances.

According to some of any of the embodiments described herein, the wall-forming material, and the fatty substance and/or plasticizer if present, are all selected in accordance with the process parameters, such that, for example, all are dissolvable, as defined herein, in a partially water-miscible organic solvent which is compatible with the microorganism and is suitable for in the encapsulation process. Exemplary such solvents are described hereinafter in the context of the process.

According to some of any of the embodiments described herein, the microcapsule is such that a viability and/or activity of the living microorganism is at least 50, or at least 60, or at least 70, or at least 80, or at least 90, %, relative to a free, non-encapsulated form of the living microorganism when tested under the same experimental conditions. Methods of determining the viability and/or activity of an encapsulated microorganism are known in the art and some are exemplified in the Examples section that follows. For example, a viability of an encapsulated microorganism can be determined by triggering a release of the encapsulated content from the microcapsule (for example, by breaking or rupturing the microcapsule) and determining viability or activity by methods known in the art.

According to some embodiments of the present invention, the microcapsule is a single-layer microcapsule.

According to some embodiments of the present invention, the microcapsule further comprises a coating layer coating the outer shell.

According to some of any of the embodiments described herein, the coating layer comprises a biocompatible polymer, a naturally-occurring polymer and/or a wall-forming material as described herein in any of the respective embodiments.

According to some embodiments of the present invention, the outer shell further comprises a fatty substance and a plasticizer, the wall-forming polymeric material comprises a naturally occurring polymer, an amount of the fatty substance ranges from about 1 to about 20% by weight of the total weight of the microcapsule; an amount of the wall-forming polymeric material ranges from about 10 to about 95% by weight of the total weight of the microcapsule; and an amount of the living microorganism ranges from about 1 to about 30% by weight of the total weight of the microcapsule.

According to some embodiments of the present invention, the outer shell further comprises a wax (e.g., a natural wax as described herein) and a plasticizer; and the wall-forming polymeric material comprises a cellulose ester.

According to some embodiments of the present invention, the outer shell further comprises a wax (e.g., a natural wax as described herein) and a plasticizer; the wall-forming polymeric material comprises a cellulose ester; an amount of the fatty substance ranges from about 5 to about 15% by weight of the total weight of the microcapsule; an amount of the wall-forming polymeric material ranges from about 15 to about 80% by weight of the total weight of the microcapsule; and an amount of the living microorganism ranges from about 5 to about 25% by weight of the total weight of the microcapsule.

According to some embodiments of the present invention, the outer shell further comprises soy wax and caprylic triglyceride; and the wall-forming polymeric material comprises cellulose acetate butyrate.

According to some embodiments of the present invention, the outer shell further comprises soy wax and caprylic triglyceride; the wall-forming polymeric material comprises cellulose acetate butyrate; an amount of the fatty substance ranges from about 5 to about 10% by weight of the total weight of the microcapsule; an amount of the wall-forming polymeric material ranges from about 20 to about 50% by weight of the total weight of the microcapsule; and an amount of the living microorganism ranges from about 10 to about 20% by weight of the total weight of the microcapsule.

### Microcapsules Composition

According to an aspect of some embodiments of the present invention there is provided a composition which comprises a plurality of microcapsules, at least a portion of the microcapsules are microcapsules which comprise an inner core comprising a living microorganism, as described herein, and an outer shell (or two or more outer shells) comprised of a wall-forming polymeric material enveloping the inner core, as described in any one of the embodiments described herein.

In some embodiments, at least 10%, at least 20%, at least 30%, at least 40%, at least 50%, at least 60%, at least 70%, at least 80%, at least 90%, at least 95%, at least 98%, or at least 99%, or substantially all of the plurality of microcapsules in the composition are microcapsules as described in any one of the embodiments described herein.

A “composition” as used herein refers to a plurality of microcapsules, which can be the same or different, which, when different, can feature a plurality or variety of features. In accordance with embodiments of the present invention, at least a portion of the plurality of microcapsules exhibits all the technical features characterizing a microorganism-encapsulating microcapsule as described herein, in any one of the embodiments thereof.

A “composition” as used in the context of these embodiments can be used as a raw material for making up a product as described herein in any of the respective embodiments.

According to an aspect of some embodiments of the present invention there is provided a plurality of microcapsules, at least a portion of the microcapsules are microcapsules which comprise an inner core comprising a living microorganism, as described herein, as described in any one of the embodiments described herein.

The plurality of microcapsules can also be referred to herein interchangeably as a mixture comprising a plurality of microcapsules.

In some embodiments, at least 10%, at least 20%, at least 30%, at least 40%, at least 50%, at least 60%, at least 70%, at least 80%, at least 90%, at least 95%, at least 98%, or at least 99%, or substantially all of the microcapsules in the plurality of microcapsules are microcapsules as described in any one of the embodiments described herein.

The term “at least a portion” means at least 20%, at least 50%, at least 70%, at least 60%. at least 80%, at least 90%, at least 95%, at least 98%, at least 99% or all of the microcapsules being the single-layer, core-shell microorganism-encapsulating microcapsules, as described in any one of the respective embodiments herein.

In some embodiments, the plurality of microcapsules as described herein can be the same, or can differ from one another by, for example, the microorganism encapsulated therein and/or the presence, absence or type of a fatty substance and/or the type of wall-forming polymeric material comprising the outer shell and/or by the presence or absence of a plasticizer and/or by the number of outer shells.

In some of the embodiments described herein for a microcapsules-containing composition or mixture which consists of microcapsules containing a living microorganism as described herein, the average size of the microcapsules is within a range of from about 10 microns to about 400 microns, or from about 10 microns to about 300 microns, or from about 10 microns to about 200 microns, or from about 10 microns to about 100 microns, or from about 50 microns to about 100 microns, including any subranges and intermediate values therebetween.

The terms “micron” and “micrometer” are used herein interchangeably.

### The Process

The process used for the preparation of the microcapsules according to embodiments of the present invention is a modification of the microencapsulation solvent removal method disclosed, for example, in U.S. Pat. Nos. 6,932,984 and 7,838,037 and WO 2012/156965, which are incorporated by reference as if fully set forth herein. According to this technology, the active ingredient is found in the core of the microcapsule. This technique seals each micro-capped ingredient from chemical and cross-link reactions, degradation, color change or loss of potency during production, and for extended periods in storage.

The solvent removal process is based on four main steps as follows:
(i) preparing a (preferably homogeneous) organic solution comprising the encapsulated agent (living microorganism), and a wall-forming polymeric material, and optionally a fatty substance and/or a plasticizer), in an organic solvent that is partially miscible in water, as defined herein;
(ii) preparing an aqueous continuous phase containing an emulsifier and saturated with the same organic solvent of the organic solution;
(iii) mixing the homogeneous organic solution with the aqueous emulsion, under high shear stirring to thereby form an emulsion; and
(iv) extracting the organic solvent by adding to the emulsion formed in step (iii) an amount of water which initiates extraction of the organic solvent from the emulsion, thereby obtaining the microcapsules.

For multi-layer (e.g., double-layer and triple-layer) microcapsules, the microcapsules are formed by first modifying the surface of the single-layer microcapsules formed according to steps (i)-(iv) and then subjecting the surface-modified inner core microcapsules to one or more cycles of steps (i)-(iv), when the inner core microcapsules are dispersed in the organic solution together with the wall-forming material.

In some embodiments, the microcapsules according to the present embodiments can be prepared a solvent removal method that comprises the following steps:
(a) contacting an organic phase comprising a living microorganism, a wall-forming polymer as described herein, and a partially water-miscible organic solvent, with an aqueous solution saturated with the organic solvent and comprising an emulsifier, to thereby obtain an emulsion; and
(b) adding to the formed emulsion an amount of water which initiates extraction of the organic solvent from the emulsion, thereby obtaining the microcapsules.

In further steps, the microcapsules are isolated following step (b), dried and sifted to thereby obtain a free flowing powder of the microcapsules.

These steps are further detailed as follows:

The homogenous solution prepared in step (a) is obtained by preparing an organic solution or dispersion of a wall-forming polymeric material as described in any one of the respective embodiments described herein, in an organic solvent that is partially miscible in water and is capable of dissolving or dispersing the wall-forming polymer. In exemplary embodiments, the organic solvent is an organic solvent that is compatible with the microorganism. In exemplary embodiments, the organic solvent is ethyl acetate.

The wall-forming polymeric material is as described in any one of the respective embodiments described herein.

According to some of any of the embodiments described herein, the organic phase further comprises a fatty substance.

According to some of any of the embodiments described herein, the organic phase further comprises a plasticizer.

According to some of any of the embodiments described herein, the organic phase further comprises a fatty substance and a plasticizer.

The fatty substance is as described in any one of the respective embodiments described herein.

The Plasticizer is as described in any one of the respective embodiments described herein.

The components of the organic phase are mixed/stirred until a homogeneous, optionally transparent, solution, suspension or dispersion is obtained.

According to some of any of the embodiments described herein, the partially water-miscible organic solvent and the wall-forming polymer are mixed at a rate of from about 200 to about 600 rpm, or from about 300 to about 500 rpm, or about 400 rpm, until complete dissolution.

According to some of any of the embodiments described herein, the partially water-miscible organic solvent and the wall-forming polymer are mixed at a temperature of about 10 to about 35° C., or about 15° C. to about 25° C., or of about 20° C., until complete dissolution.

According to some of any of the embodiments described herein, the partially water-miscible organic solvent, the wall-forming polymer, and the living microorganism are mixed at a rate of about 100 to about 500 rpm, or from about 150 to about 350 rpm, or about 250 rpm, at room temperature.

According to some of any of the embodiments described herein, the partially water-miscible organic solvent, the wall-forming polymer, and the living microorganism are mixed for at least 1 minute, or at least 3 minutes, or about 5 minutes.

According to some of any of the embodiments described herein, the emulsion is mixed at a rate of about 100 to about 500 rpm, or from about 150 to about 350 rpm, or about 250 rpm.

According to some of any of the embodiments described herein, the emulsion is mixed for at least 30 seconds, or at least 1 minute, or at least 2 minutes.

The aqueous continuous phase is saturated with the organic solvent that forms the organic solution, and typically comprises an emulsifier.

The organic solution or dispersion and the aqueous continuous phase are mixed under low sheer stirring to thereby form an emulsion. The addition of water results in intra-surface solidification of the oil droplets containing the dispersed probiotic microorganisms.

In step (b), an amount of water is added to the emulsion prepared in (a), thereby extracting the organic solvent and allowing the microcapsules to form. At this stage the organic solvent immigrates to the external water phase and solid microcapsules are obtained. The microcapsules are left for complete sedimentation.

In the context of embodiments of the invention, the term “low sheer stirring” refers to a mixing at about 100-800 rpm, preferably at about 300-600 rpm.

In some embodiments, when the microcapsules are multi-layer microcapsules, the process further comprises: (c) optionally repeating steps (a) and (b), using a second, third, and so on, organic phases and aqueous continuous phases, thereby obtaining multi-layered microcapsules.

The obtained microcapsules are then washed and filtered to remove residues of the solvent.

According to some of any of the embodiments described herein, the process further comprises dehydrating the microcapsules to thereby obtain dehydrated microcapsules.

The dehydration can be effected, for example, by freeze drying, heat drying, vacuum drying or any combination thereof.

The obtained dehydrated microcapsules are in the form of a powder.

According to some embodiments, the dehydration is effected by freeze-drying (lyophilization). This drying technique is a dehydration process which works by freezing the product and then reducing the surrounding pressure to allow the frozen water to sublimate directly from the solid phase to the gas phase. The process is performed by freezing the encapsulated microorganism (e.g., probiotics) in the presence of carrier material at low temperatures, followed by sublimation of the water under vacuum, such that water phase transition and oxidation are avoided. In order to improve the microorganism (e.g., probiotic) activity upon freeze-drying and also stabilize them during storage, cryoprotectants can be added.

The final water content in the microcapsule can be pre-determined as needed. A higher water content provides for a higher survival rate after the drying process, and a lower water content provides for lower inactivation during storage.

### Products and Uses

As described herein, the microorganism-containing microcapsules can be incorporated in various products that can benefit from the activity of encapsulated microorganism.

Such products include, but are not limited to, a pharmaceutical product which comprises microorganisms having a therapeutic effect, as described herein; a cosmetic product which comprises microorganisms having a cosmetic effect, as described herein; a cosmeceutical product which comprises microorganisms having a cosmeceutical effect, as described herein; a food product which comprises microorganisms having a beneficial physiological effect, as described herein; and an agricultural product which comprises microorganisms having an agricultural effect, as described herein.

According to some of any of the embodiments described herein, the microorganism-encapsulating microcapsules as described herein are usable for inclusion in topical formulations and products.

In some embodiments, the composition provided herein is used in cosmetic, cosmeceutical or pharmaceutical formulations such as skin care formulations, make-up or dermatological or other topical pharmaceutical formulations, comprising the microcapsules as described herein. The formulation can optionally and preferably further comprise a carrier, and optionally additional active agents and/or additives.

As used herein a “formulation” refers to a vehicle in the form of emulsion, lotion, cream, gel, powder, etc., that comprises the microorganism-encapsulating microcapsules as described herein with physiologically acceptable carriers and excipients and optionally other chemical components such as cosmetic, cosmeceutical or pharmaceutical agents (e.g., drugs).

As used herein, the term “physiologically acceptable” means approved by a regulatory agency of the Federal or a state government or listed in the U.S. Pharmacopeia or other generally recognized pharmacopeia for use in animals, and more particularly in humans.

Herein, the phrase “physiologically suitable carrier” refers to an approved carrier or a diluent that does not cause significant irritation to an organism and does not abrogate the biological activity and properties of a possible active agent.

Herein the term “excipient” refers to an inert substance added to a pharmaceutical composition to further facilitate processes and administration of the active ingredients.

In some embodiment of the present invention, the cosmetic or cosmeceutical formulation is formulated in a form suitable for topical application on the applied area (e.g., facial skin).

By selecting the appropriate carrier and optionally other ingredients that can be included in the composition, as is detailed hereinbelow, the compositions of the present embodiments may be formulated into any form typically employed for topical application.

By “appropriate carrier” for topical application it is meant any medium compatible with a keratinous substrate, which has a color, a smell and a pleasant feel and which does not generate unacceptable discomfort (stinging, tautness or redness).

The phrase “keratinous material” or “keratinous substrate” means, in some embodiments of the present invention, the skin and especially areas like the face, checks, hands, body, legs, around the eyes, the eyelids and the lips.

The formulations can be water-based, oil-based, emulsion-based (including water-in-oil, oil-in-water, water-in-oil-in-water and oil-in-water-in-oil emulsions) or silicon-based.

The formulations as described herein can be, for example, skin care products, make-up products (including eye shadows, make-up, lipstick, lacquer, etc., or any other product as described herein).

In some embodiments, a formulation as described is in a form of a cream, an ointment, a paste, a gel, a lotion, a milk, an oil, a suspension, a solution, an aerosol, a spray, a foam, a powder (e.g., a pressed powder or a loose powder) or a mousse.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emolliency). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the sunscreens-containing microcapsules, are present in a water or alcohol base. Lotions are typically preferred for covering/protecting large body areas, due to the case of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.c., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or hydroalcoholic, but are typically formulated with high alcohol content which, upon application to the skin of a user, quickly evaporates, driving the active ingredient through the upper skin layers to the site of treatment.

The preparation of the formulation can be carried out by mixing and homogenizing all the ingredients except for the microorganism-encapsulating microcapsules, and adding the microorganism-encapsulating microcapsules at the end, followed by low shear mixing of the mixture.

The microorganism-encapsulating microcapsules of the invention can be used in pharmaceutical compositions for topical application, which include, for example, pharmaceutically active agents for dermatological or transdermal applications.

In any of the formulations described herein, additional agents and/or additives can be included. These agents and/or additives and can be encapsulated or non-encapsulated.

In some embodiments, one or more of these agents and/or additives is encapsulated.

In some of these embodiments, the agents and/or additives are encapsulated using microcapsules as described in any one of U.S. Pat. Nos. 6,932,984 and 7,838,037, and WO 2009/138978.

Some non-limiting representative examples of additives and/or agents include humectants, deodorants, antiperspirants, sunscreen agents (e.g., UV blocking agents, UV filters), sunless tanning agents, hair conditioning agents, pH adjusting agents, chelating agents, preservatives, emulsifiers, occlusive agents, emollients, thickeners, solubilizing agents, penetration enhancers, anti-irritants, colorants, propellants and surfactants.

Representative examples of humectants include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propyleneglycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

Suitable pH adjusting agents include, for example, one or more of adipic acids, glycines, citric acids, calcium hydroxides, magnesium aluminometasilicates, buffers or any combinations thereof.

Representative examples of deodorant agents include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmithyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate. Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the compositions of the present invention either in a solubilized or a particulate form and include, for example, aluminum or zirconium astringent salts or complexes.

Representative examples of sunless tanning agents include, without limitation, dihydroxyacetone, glyceraldehyde, indoles and their derivatives. The sunless tanning agents can be used in combination with the sunscreen agents.

The chelating agents are optionally added to formulations so as to enhance the preservative or preservative system. Preferred chelating agents are mild agents, such as, for example, ethylenediaminetetraacetic acid (EDTA), EDTA derivatives, or any combination thereof.

Suitable preservatives include, without limitation, one or more alkanols, disodium EDTA (ethylenediamine tetraacetate), EDTA salts, EDTA fatty acid conjugates, isothiazolinone, parabens such as methylparaben and propylparaben, propyleneglycols, sorbates, urea derivatives such as diazolindinyl urea, or any combinations thereof.

Suitable emulsifiers include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients include, for example, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, fatty acids and their alkali salts and mixtures thereof.

Representative examples of solubilizing agents that are usable in this context of the present invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEENS and spans, e.g., TWEEN® 80. Other solubilizers that are usable for the compositions of the present invention are, for example, polyoxyethylene sorbitan fatty acid ester, polyoxyethylene n-alkyl ethers, n-alkyl amine n-oxides, poloxamers, organic solvents, phospholipids and cyclodextrines.

Suitable penetration enhancers include, but are not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propyleneglycol (PG), propyleneglycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, and the like. The permeation enhancer may also be a vegetable oil. Such oils include, for example, safflower oil, cottonseed oil and corn oil.

Suitable anti-irritants include, for example, steroidal and non-steroidal anti-inflammatory agents or other materials such as aloe vera, chamomile, alpha-bisabolol, cola nitida extract, green tea extract, tea tree oil, licoric extract, allantoin, caffeine or other xanthines, glycyrrhizic acid and its derivatives.

Exemplary additional active agents according to these embodiments of present invention include, without limitation, one or more, or any combination of an antibiotic agent, an antimicrobial agent, an anti-acne agent, an anti-aging agent, a wrinkle-reducing agent, a skin whitening agent, a sebum reducing agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, an antineoplastic agent, an immunomodulator, an interferon, an antidepressant, an anti-histamine, a vitamin, a hormone and an anti-dandruff agent.

In some of any of the embodiments described herein, a topical formulation comprises, in addition to the microcapsules encapsulating a living microorganism as described herein, an additional agent, as described herein, which is encapsulated. In some of these embodiments, the topical formulation comprises an additional type of microcapsules, which encapsulate the additional agent. In some of these embodiments, the additional type of microcapsules is selected as being compatible with the microcapsules encapsulating a living microorganism agent as described herein.

Exemplary such microcapsules are microcapsules as described in U.S. Pat. Nos. 6,932,984 and 7,838,037 and WO 2012/156965. More specific examples include microcapsules marketed by Tagra, under the tradenames TagraCap1™, TagraCap3™ and CameleonCaps™, for colorant-encapsulating microcapsules; SunCaps™, for UV filter-encapsulating microcapsules; Tagravit™ and CelluCap™ for vitamin-encapsulating microcapsules; and Tagrol™ and CelluOil™ for essential oil-encapsulating microcapsules.

Alternatively or addition, the microorganism-encapsulating microcapsules as described herein are usable in making up a pharmaceutical product, that is a pharmaceutical composition, or a drug. The pharmaceutical product or composition can be formulated as a topical formulation as described herein, or for systemic or local administration.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to an agent or a substance accountable for the biological effect. In some embodiments, the active ingredient or agent is or comprises the microorganism-encapsulating microcapsules as described herein. In some embodiments, additional agents that exhibit a therapeutic effect are included in the pharmaceutical composition or product.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For topical administration, any of the topical formulations as described herein are contemplated.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using. e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein, the term “subject” includes mammals, preferably human beings at any age which suffer from the pathology. Preferably, this term encompasses individuals who are at risk to develop the pathology.

Further alternatively or in addition, the microorganism-encapsulating microcapsules of the present embodiments can be included in a food or any other edible product, and the microcapsules encapsulate a living microorganism that has a beneficial effect in a food product.

As used herein, the phrase “food product” describes an edible product consisting essentially of protein, carbohydrate and/or fat, which is used in the body of an organism to sustain growth, repair and vital processes and to furnish energy. Food products may also contain supplementary substances such as minerals, vitamins and condiments. See Merriam-Webster's Collegiate Dictionary, 10th Edition, 1993. The phrase “food product” as used herein further includes a beverage adapted for human or animal consumption.

A food product containing the microorganism-encapsulating microcapsules can also include additional additives such as, for example, antioxidants, sweeteners, flavorings, colors, preservatives, enzymes, nutritive additives such as vitamins and minerals, emulsifiers, pH control agents such as acidulants, hydrocolloids, antifoams and release agents, flour improving or strengthening agents, raising or leavening agents, gases and chelating agents, the utility and effects of which are well-known in the art.

The microorganism-encapsulating microcapsules can also be included in an agricultural product or formulation such as, for example, a product or formulation usable to promote growth or protect a crop, which is also referred to in the art as an agrochemical, including, for example, pesticides, fertilizers, hormones and other chemical growth agents. The microcapsules encapsulate a living microorganism that has a beneficial effect in such a product.

An agrochemical typically further comprises an agriculturally acceptable carrier.

Examples of agriculturally acceptable carriers and formulations are disclosed in U.S. Patent Application Publication Nos. 2018/0271088, 2009/0148399, 2015/0257378, the contents of which are incorporated herein by reference.

The agricultural formulation can be in the form of a liquid (e.g., a homogeneous liquid or an emulsion), a semi-solid (e.g., a paste, a gel), or a solid (e.g., a rubber, a glass, a sol-gel).

Exemplary carriers include oils, water-in-oil emulsions or oil-in-water emulsions; a solid substrate such as fibers (e.g., cotton fibers, felts); polymers (e.g., polyethylene glycol, polymethacrylates, ethylene-vinyl acetate rubbery copolymers, poly(acrylic acid), polyolefins (e.g., polypropylene), poly(urethane), silicones, lactic and glycolic acid-based polymers, and copolymers thereof); a gel; and ceramics.

Exemplary oils include, but are not limited to, oils derived from plants such as vegetable oils and nut oils, or non-plant derived oils such as mineral oils. These are widely available and cost-effective. Formulations can include oils such as canola oil, cottonseed oil, palm oil, safflower oil, soybean oil, corn oil, olive oil, peanut oil, sunflower oil, sesame oil, nut oils, and coconut oils. Nut oils include, but are not limited to, almond oil, cashew oil, hazelnut oil, macadamia oil, mongongo nut oil, pecan oil, pine nut oil, pistachio oil, sacha inchi oil, and walnut oil. The oils can be used per se or in a form of an emulsion along with an aqueous phase.

The agricultural product can comprise, in addition to the respective microcapsules, additional active agents (e.g., additional agrochemicals as described herein).

It is expected that during the life of a patent maturing from this application many relevant probiotics will be developed and the scope of the term probiotic is intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Microencapsulation

Cellulose acetate butyrate polymers having butyryl content of 17%, hydroxyl content of 1.5%, or having butyryl content of 37%, acetyl content of 13%, and hydroxyl content of 1.5% and molecular weight (Mn) of about 20,000 grams/mol were used.

Other reagents were obtained from commercial vendors.

Table 1 below summarizes exemplary raw materials used to form 50 grams microorganism-containing microcapsules.

TABLE 1

| Organic phase | | | Emulsion | | | Extraction | | |
|---|---|---|---|---|---|---|---|---|
| | | Amount | | | Amount | | | Amount |
| Ingredient | % | (grams) | Ingredient | % | (grams) | Ingredient | % | (grams) |
| Ethyl | | 200 | Water | | 512 | Water | | 5900 |
| acetate | | | | | | | | |
| Cellulose | 30 | 15 | Ethyl | 10 | 65 | PVA (4%) | 0.2 | 347 |
| Acetate | | | acetate | | | | | |
| Butyrate | | | | | | | | |
| Microbiome | 15 | 7.5 | PVA (4%) | 0.5 | 73 | Emulsion | | 900 |
| strain | | | | | | | | |
| Soy wax | 5 | 2.5 | Organic | | 250 | | | |
| | | | phase | | | | | |
| Caprylic | 50 | 25 | | | | | | |
| triglyceride | | | | | | | | |

The wall-forming polymer cellulose acetate butyrate (CAB) was dissolved in ethyl acetate at 20° C. using paddle mixer at 400 rpm following the addition of soy wax and caprylic triglyceride until complete dissolution. A powder of a probiotic strain was then added and mixed for additional 5 minutes.

After 5 minutes the organic phase was transferred to an aqueous phase containing 10% ethyl acetate while mixing at 250 rpm for 5 minutes. 65 grams water was then added to the emulsion to start the intra-surface solidification process. After 2 minutes of additional mixing the emulsion was added to 5900 grams water for complete solidification while mixing for 15 minutes at 240 rpm. The obtained microcapsules were then left for sedimentation for 3 hours at 10° C., and were thereafter filtered.

The microcapsules were then subjected to freeze drying for 48 hours at a maximal temperature of 25° C.

### Example 2

### Characterization and Activity

### Morphology and Particle Size:

As can be seen in FIGS. 1A-B, the dried microcapsules exhibit spherical shape with median particle size of about 60 microns.

### Microbiome Activity:

In order to demonstrate the longevity and activity of the probiotic microorganism after encapsulation, the obtained dry microcapsules were transferred into a water-containing plastic tube. Same was done with the non-encapsulated probiotic sample.

Since the probiotic eventually produce lactic acid, a reduction in pH should be observed.

The pH over time (right after adding the sample to water) was measured in comparison to the pH at time zero.

As can be seen in FIG. 2, once the free probiotic microorganism was added to water, the pH was immediately reduced by 2.5 units.

The encapsulated form of the probiotic did not cause any change in the pH over time. When the microcapsules were subjected to conditions that cause their breakage (using homogenizer, vortex and Tween® 80), the pH of the aqueous solution was reduced over 4 days, indicating that the probiotic microorganism remained viable and maintained its activity during the encapsulation process.

Samples of encapsulated probiotic after breakage using homogenization, vortex and Tween® 20 were planted on an agar plate and development of new colonies were observed, further demonstrating the maintained viability of the probiotic microorganism following the encapsulation process.

### Example 3

### Viability of Microencapsulated Probiotics

In order to examine the compatibility of probiotic microorganisms in the exemplary organic solvent used in the microencapsulation process, the viability of two exemplary probiotic strains, L. plantarum and L. acidofulus, was determined in ethyl acetate.

Each probiotic strain was added to ethyl acetate solution, vortexed for 30 seconds and incubated for additional 10 minutes. The samples were then diluted to appreciate concentration and pour-plated in MRS agar. The plates were incubated at 37° C. for 48 hours under anaerobic condition.

The viable cell number is expressed as colony forming unit per gram of microcapsule (CFU/g). Surprisingly, as FIG. 3 shows, both L. plantarum and L. acidofulus had demonstrated high viability following treatment with ethyl acetate.

In order to further examine the viability of the encapsulated probiotics following the encapsulation process and the durability of the microcapsules, the viability of L. plantarum in L. plantarum-loaded microcapsules was determined in the presence of Tween® 80.

To a 50 mL plastic tube, 10 mL of MRS agar medium and 500 μL Tween® 80 were added. The sample was incubated for 20 minutes at 40° C. while shaking. Then, 2 grams of L. plantarum-loaded microcapsules were added and incubated while shaking for additional 2 hours at 40° C. Samples were then diluted to appreciate concentration and pour-plated in MRS agar. The plates were incubated at 37° C. for 48 hours under anaerobic condition. As FIG. 4 shows, each gram of microcapsules was revealed to be loaded with about 93,000 CFU of L. plantarum, demonstrating the protecting effect of microcapsules.

The shape and loading of the microencapsulated probiotics was monitored using a SEM analysis, and as FIGS. 5A and 5B present, L. plantarum-loaded microcapsules powder raw material exhibit a spherical shape, and do not contain any free-form L. plantarum.

Overall, the probiotic strains were found to be compatible with the exemplary organic solvent which was being used in the encapsulation process.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A microcapsule comprising an inner core enveloped by an outer shell formed of a wall-forming polymeric material, said inner core comprises a living microorganism, the microcapsule being characterized by:
(a) said wall-forming polymeric material having a positive Log P value; and/or
(b) the microcapsule is rupturable by a mechanical action of rubbing or pressing with a human hand; and/or
(c) said wall-forming polymeric material being dissolvable at a concentration of at least 100 grams per liter in a partially water-miscible organic solvent that is compatible with said microorganism.

2. The microcapsule of claim 1, characterized by said (a), (b) and (c).

3. The microcapsule of claim 1, wherein said outer shell further comprises a fatty substance.

4. (canceled)

5. The microcapsule of claim 3, wherein said fatty substance is a naturally occurring and/or biocompatible fatty substance.

6. The microcapsule of claim 5, wherein said fatty substance is a natural wax.

7-9. (canceled)

10. The microcapsule of claim 1, wherein an amount of said living microorganism ranges from about 1 to about 30, or from about 5 to about 25, or from about 10 to about 20, % by weight of the total weight of the microcapsule.

11. The microcapsule of claim 1, wherein said outer shell further comprises a plasticizer.

12. The microcapsule of claim 11, wherein said plasticizer is a naturally-occurring and/or biocompatible plasticizer.

13-14. (canceled)

15. The microcapsule of claim 1, wherein said wall-forming polymer comprises a naturally occurring polymer and/or biocompatible polymer.

16. (canceled)

17. The microcapsule of claim 1, wherein said wall-forming polymer comprises a cellulose ester.

18. The microcapsule of claim 17, wherein said cellulose ester comprises from 1 to 10% free hydroxy groups.

19. The microcapsule of claim 17, wherein said wall-forming polymer is or comprises cellulose acetate butyrate.

20. (canceled)

21. The microcapsule of claim 1, wherein said wall-forming polymeric material has a log P value in a range of from 2 to 20, or from 2 to 10 and/or is dissolvable in ethyl acetate at a concentration of from 100 grams to 1,000 grams per liter, or from 100 grams to 800 grams per liter.

22. (canceled)

23. The microcapsule of claim 1, wherein said living microorganism comprises a probiotic microorganism.

24. The microcapsule of claim 23, wherein said probiotic microorganism is beneficial for use in a pharmaceutical product, a cosmetic product, a cosmeceutical product, a food product, or an agricultural product.

25-28. (canceled)

29. The microcapsule of claim 1, wherein a viability and/or activity of said living microorganism is at least 50, or at least 60, or at least 70, or at least 80, or at least 90, %, relative to a free, non-encapsulated form of said living microorganism when tested under the same experimental conditions.

30. The microcapsule of claim 1, being a single-layer microcapsule.

31. The microcapsule of claim 1, wherein:
said outer shell further comprises a fatty substance and a plasticizer,
said wall-forming polymeric material comprises a naturally occurring polymer,
and wherein:
an amount of said fatty substance ranges from about 1 to about 20% by weight of the total weight of the microcapsule;
an amount of said wall-forming polymeric material ranges from about 10 to about 95% by weight of the total weight of the microcapsule; and
an amount of said living microorganism ranges from about 1 to about 30% by weight of the total weight of the microcapsule.

32. The microcapsule of claim 1, wherein:
said outer shell further comprises a wax and a plasticizer;
said wall-forming polymeric material comprises a cellulose ester;
and wherein:
an amount of said fatty substance ranges from about 5 to about 15% by weight of the total weight of the microcapsule;
an amount of said wall-forming polymeric material ranges from about 15 to about 80% by weight of the total weight of the microcapsule; and
an amount of said living microorganism ranges from about 5 to about 25% by weight of the total weight of the microcapsule.

33. The microcapsule of claim 1, wherein:
said outer shell further comprises soy wax and caprylic triglyceride;
said wall-forming polymeric material comprises cellulose acetate butyrate;
and wherein:
an amount of said fatty substance ranges from about 5 to about 10% by weight of the total weight of the microcapsule;
an amount of said wall-forming polymeric material ranges from about 20 to about 50% by weight of the total weight of the microcapsule; and
an amount of said living microorganism ranges from about 10 to about 20% by weight of the total weight of the microcapsule.

34. A composition comprising a plurality of microcapsules, wherein in at least a portion of said microcapsules, each microcapsule is a microcapsule according to claim 1.

35. A product comprising the composition of claim 34.

36. (canceled)

37. A process of preparing the microcapsule according to claim 1, the process comprising:
(a) contacting an organic phase comprising said partially water-miscible organic solvent, said wall-forming polymer, said living microorganism, and an emulsifier, with an aqueous continuous phase saturated with said partially water-miscible organic solvent, to thereby obtain an emulsion; and
(b) adding to said emulsion an amount of water which initiates extraction of said partially water-miscible organic solvent from said emulsion, thereby obtaining the microcapsules encapsulating said living microorganisms.

38. The process of claim 37, wherein said partially water-miscible organic solvent is compatible with said microorganism.

39. (canceled)

40. The process of claim 37, wherein said organic phase further comprises a fatty substance.

41-44. (canceled)

45. The process of claim 37, wherein said organic phase further comprises a plasticizer.

46. The process of claim 37, further comprising isolating the microcapsules.

47. (canceled)

48. The process of claim 46, further comprising dehydrating the microcapsules to obtain dehydrated microcapsules.

49. (canceled)

50. The process of claim 48, wherein said dehydrated microcapsules are in the form of a powder.
